SEQUENCE LISTING (7 sequences)

SEQ ID NO: 1 - DNA, 1519 bp, Megasphaera massiliensis
  Megasphaera massiliensis gene for 16S ribosomal RNA, partial sequence, strain NP3 - JX424772.1
agagtttgatcctggctcaggacgaacgctggcggcgtgcttaacacatgcaagtcgaacgagaagagatgagaagcttgcttcttatcaattcgagtggcaaacgggtgagtaacgcgtaagcaacctgcccttcagatggggacaacagctggaaacggctgctaataccgaatacgttctttccgccgcatgacgggaagaagaaagggaggccttcgggctttcgctggaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatatgggacgaacagggcatcggttaatacccggtgtctttgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggacgcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaaggaaccagccgtcgaaggtgggggcgatgattggggtgaagtcgtaacaaggt

SEQ ID NO: 2 - DNA, 1398 bp, Megasphaera massiliensis
  consensus 16S rRNA sequence for Megasphaera massiliensis strain 42787
tgagaagcttgcttcttatcgattctagtggcaaacgggtgagtaacgcgtaagcaacctgcccttcagatggggacaacagctggaaacggctgctaataccgaatacgttctttccgccgcatgacgggaagaagaaagggaggccttcgggctttcgctggaggaggggcttgcgtctgattagctagttggaggggtaacggcccaccaaggcgacgatcagtagccggtctgagaggatgaacggccacattgggactgagacacggcccagactcctacgggaggcagcagtggggaatcttccgcaatggacgaaagtctgacggagcaacgccgcgtgaacgatgacggccttcgggttgtaaagttctgttatatgggacgaacaggacatcggttaatacccggtgtctttgacggtaccgtaagagaaagccacggctaactacgtgccagcagccgcggtaatacgtaggtggcaagcgttgtccggaattattgggcgtaaagggcgcgcaggcggcatcgcaagtcggtcttaaaagtgcggggcttaaccccgtgaggggaccgaaactgtgaagctcgagtgtcggagaggaaagcggaattcctagtgtagcggtgaaatgcgtagatattaggaggaacaccagtggcgaaagcggctttctggacgacaactgacgctgaggcgcgaaagccaggggagcaaacgggattagataccccggtagtcctggccgtaaacgatggatactaggtgtaggaggtatcgactccttctgtgccggagttaacgcaataagtatcccgcctggggagtacggccgcaaggctgaaactcaaaggaattgacgggggcccgcacaagcggtggagtatgtggtttaattcgacgcaacgcgaagaaccttaccaagccttgacattgattgctacggaaagagatttccggttcttcttcggaagacaagaaaacaggtggtgcacggctgtcgtcagctcgtgtcgtgagatgttgggttaagtcccgcaacgagcgcaacccctatcttctgttgccagcacctcgggtggggactcagaagagactgccgcagacaatgcggaggaaggcggggatgacgtcaagtcatcatgccccttatggcttgggctacacacgtactacaatggctcttaatagagggaagcgaaggagcgatccggagcaaaccccaaaaacagagtcccagttcggattgcaggctgcaactcgcctgcatgaagcaggaatcgctagtaatcgcaggtcagcatactgcggtgaatacgttcccgggccttgtacacaccgcccgtcacaccacgaaagtcattcacacccgaagccggtgaggcaaccgcaag

SEQ ID NO: 3 - DNA, 1409 bp, Megasphaera
  consensus 16S rRNA sequence for the Megasphaera strain deposited under accession number NCIMB 43385
ggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatc

SEQ ID NO: 4 - DNA, 1415 bp, Megasphaera massilliensis
  consensus 16S rRNA sequence for the Megasphaera massilliensis strain deposited under accession number NCIMB 43388
ggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccgaggtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcaaagacaccgggtattaaccgatgtcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcttcccgtcatgcggcggaaagaacgtattcggtattagcagccgtttccagctgttgtccccatctgaagggcaggttgcttacgcgttactcacccgtttgccactagaatcgataagaagcaagcttctcatgtcttct

SEQ ID NO: 5 - DNA, 1433 bp, Megasphaera massilliensis
  consensus 16S rRNA sequence for the Megasphaera massilliensis strain deposited under accession number NCIMB 43389
cgacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccgaggtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcaaagacaccgggtattaaccgatgccctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaaccagctaatcagacgcaagcccctcctccagcgaaagcccgaaggcctccctttcttcttcccgtcatgcggcggaaagaacgtattcggtattagcagccgtttccagctgttgtccccatctgaagggcaggttgcttacgcgttactcacccgtttgccactagaatcgataagaagcaagcttctcatgtcttctcgttcgacttgcat

SEQ ID NO: 6 - DNA, 1431 bp, Megasphaera
  consensus 16S rRNA sequence for the Megasphaera strain deposited under accession number NCIMB 43386
cgacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatctcttctcgttcgactgca

SEQ ID NO: 7 - DNA, 1434 bp, Megasphaera
  consensus 16S rRNA sequence for the Megasphaera strain deposited under accession number NCIMB 43387
tcgaacggctggttccttgcggttgcctcaccggcttcgggtgtgaatgactttcgtggtgtgacgggcggtgtgtacaaggcccgggaacgtattcaccgcagtatgctgacctgcgattactagcgattcctgcttcatgcaggcgagttgcagcctgcaatccgaactgggactctgtttttggggtttgctccggatcgctccttcgcttccctctattaagagccattgtagtacgtgtgtagcccaagccataaggggcatgatgacttgacgtcatccccgccttcctccgcattgtctgcggcagtctcttctgagtccccacccttagtgctggcaacagaagataggggttgcgctcgttgcgggacttaacccaacatctcacgacacgagctgacgacagccgtgcaccacctgttttcttgtcttccgaagaagaaccggaaatctctttccgtagcaatcaatgtcaaggcttggtaaggttcttcgcgttgcgtcgaattaaaccacatactccaccgcttgtgcgggcccccgtcaattcctttgagtttcagccttgcggccgtactccccaggcgggatacttattgcgttaactccggcacagaaggagtcgatacctcctacacctagtatccatcgtttacggccaggactaccggggtatctaatcccgtttgctcccctggctttcgcgcctcagcgtcagttgtcgtccagaaagccgctttcgccactggtgttcctcctaatatctacgcatttcaccgctacactaggaattccgctttcctctccgacactcgagcttcacagtttcggtcccctcacggggttaagccccgcacttttaagaccgacttgcgatgccgcctgcgcgccctttacgcccaataattccggacaacgcttgccacctacgtattaccgcggctgctggcacgtagttagccgtggctttctcttacggtaccgtcagggataacgggtattgaccgctatcctgttcgtcccatataacagaactttacaacccgaaggccgtcatcgttcacgcggcgttgctccgtcagactttcgtccattgcggaagattccccactgctgcctcccgtaggagtctgggccgtgtctcagtcccaatgtggccgttcatcctctcagaccggctactgatcgtcgccttggtgggccgttacccctccaactagctaatcagacgcaagcccctcctccagcgaaagcccgaaggcctccctttcttcatcccgtcatgcggcggaaagaacgtattcggtattagcagccgtttccagctgttgtccccatctgaagggcaggttgcttacgcgttactcacccgtttgccactcgaattgataagaagcaagcttctcatctcttctcgttcgacttgca

(19) United States
(12) Patent Application Publication
(10) Pub. No.: US 2022/0054558 A1
(43) Pub. Date: Feb. 24, 2022

(54) COMPOSITIONS COMPRISING BACTERIAL STRAINS

(71) Applicant: 4D Pharma Research Limited, Aberdeen (GB)

(72) Inventors: Alexander Stevenson, Aberdeen (GB); Samantha Yuille, Aberdeen (GB)

(21) Appl. No.: 17/342,578

(22) Filed: Jun. 9, 2021

Related U.S. Application Data
(63) Continuation of application No. PCT/EP2019/084993, filed on Dec. 12, 2019.

(30) Foreign Application Priority Data
Dec. 12, 2018 (EP) 18212006.3
Dec. 19, 2018 (GB) 1820745.6
May 13, 2019 (GB) 1906732.1
Oct. 14, 2019 (GB) 1914856.8

(51) Int. Cl. A61K 35/74; C12N 1/20

(52) U.S. Cl. CPC A61K 35/74; C12N 1/205

(57) ABSTRACT

The invention provides a composition comprising a strain of a bacteria for use in the treatment or prevention of a Gram-positive bacterial infection in a subject, wherein the strain produces valeric acid.

### CROSS-REFERENCE

This application is a continuation of International Application No. PCT/EP2019/084993, filed Dec. 12, 2019, which claims the benefit of Great Britain Application No. 1820745.6, filed Dec. 19, 2018, Great Britain Application No. 18212006.3, filed Dec. 12, 2018, Great Britain Application No. 1906732.1, filed May 13, 2019, and Great Britain Application No. 1914856.8, filed Oct. 14, 2019, all of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Jun. 2, 2021, is named 56708_755_301_SL.txt and is 14,076 bytes in size.

### TECHNICAL FIELD

This invention is in the field of compositions comprising bacterial strains isolated from the mammalian digestive tract and the use of such compositions in the treatment of disease.

### BACKGROUND TO THE INVENTION

The human intestine is thought to be sterile in utero, but it is exposed to a large variety of maternal and environmental microbes immediately after birth. Thereafter, a dynamic period of microbial colonization and succession occurs, which is influenced by factors such as delivery mode, environment, diet and host genotype, all of which impact upon the composition of the gut microbiota, particularly during early life. Subsequently, the microbiota stabilizes and becomes adult-like [1]. The human gut microbiota contains more than 500-1000 different phylotypes belonging essentially to two major bacterial divisions, the Bacteroidetes and the Firmicutes [2]. The successful symbiotic relationships arising from bacterial colonization of the human gut have yielded a wide variety of metabolic, structural, protective and other beneficial functions. The enhanced metabolic activities of the colonized gut ensure that otherwise indigestible dietary components are degraded with release of by-products providing an important nutrient source for the host. Similarly, the immunological importance of the gut microbiota is well-recognized and is exemplified in germfree animals which have an impaired immune system that is functionally reconstituted following the introduction of commensal bacteria [3-5].

Dramatic changes in microbiota composition have been documented in gastrointestinal disorders such as inflammatory bowel disease (IBD). For example, the levels of Clostridium cluster XIVa bacteria are reduced in IBD patients whilst numbers of E. coli are increased, suggesting a shift in the balance of symbionts and pathobionts within the gut [6-9].

In recognition of the potential positive effect that certain bacterial strains may have on the animal gut, various strains have been proposed for use in the treatment of various diseases (see, for example, [10-13]). Also, certain strains, including mostly Lactobacillus and Bifidobacterium strains, have been proposed for use in treating various inflammatory and autoimmune diseases that are not directly linked to the intestines (see [14] and [15] for reviews). However, the relationship between different diseases and different bacterial strains, and the precise effects of particular bacterial strains on the gut and at a systemic level and on any particular types of diseases are poorly characterised.

A “healthy” microbiome is also known to suppress colonization of the gut by pathogenic Gram-positive bacteria. Gram-positive bacteria are characterised by giving a positive result in the Gram-stain test [16]. They are generally characterised as having cytoplasmic lipid membranes and a thick outer peptidoglycan chains cross-linked to form a rigid cell wall. Numerous pathogenic strains of Gram-positive have been identified that cause a range of infections in humans and other subjects. In particular, the Gram-positive bacteria C. difficile is the most common cause of antibiotic-associated diarrhea in hospitals and other healthcare facilities. The elderly are particularly susceptible and at increased risk for adverse outcome as a result of C. difficile infection [17].

C. difficile resides in the gastrointestinal tract asymptomatically in 2%-5% of the population, however in subjects at risk, such as the elderly or those with weakened immune systems or those who have undergone or are undergoing antibiotic therapy, pathological expansion of C. difficile can occur, which can lead to a variety C. difficile associated diseases (CDAD), ranging from mild diarrhea to severe colitis and toxic megacolon.

The mechanisms underlying pathological expansion of Gram-positive bacteria such as C. difficile remain unknown, but C. difficile infections can be treated with antibiotics and to some extent by faecal matter transplant (FMT) therapy. Subjects remain predisposed to recurrent infections and although FMT has been touted as an effective anti-recurrent infection treatment option, FMT is a non-standardized procedure, and the long-term consequences of transplanting donor faecal matter into a host's gastrointestinal tract remains unknown [18]. There is a need therefore to develop therapies for the treatment of Gram-positive bacterial infections, such as C. difficile infection.

### SUMMARY OF THE INVENTION

The inventors have developed new therapies for treating and preventing Gram-positive bacterial infections in a subject. In particular, the inventors have identified that bacteria that produce valeric acid are effective for killing Gram-positive bacteria. In addition, valeric acid has been shown to reduce the viability of pathogenic Gram-positive bacteria [19]. Therefore, the compositions of the invention may be particularly effective for use in the treatment or prevention of pathogenic Gram-positive bacterial infections.

The examples show that a commensal bacterial strain that produces valeric acid kills the Gram-positive bacteria Bacillus subtilis. In some embodiments, therefore, the composition of the invention is for use in reducing the viability of a Gram-positive bacteria in the treatment of prevention of a Gram-positive bacterial infection. In other words, the composition has cytotoxic activity with respect to the Gram-positive bacteria causing the infection. The examples show that a commensal bacterial strain that produces valeric acid inhibits the growth of the Gram-positive bacteria Bacillus subtilis. In some embodiments, therefore, the composition of the invention is for use in inhibiting the growth of a Gram-positive bacteria in the treatment of prevention of the Gram-positive bacterial infection. In other words, the composition is cytostatic activity with respect to the Gram-positive bacteria.

The bacteria of the invention may be used to restore the level of pathogenic Gram-positive bacteria to asymptomatic levels or to eliminate the pathogenic Gram-positive bacteria entirely from a subject, thereby treating the Gram-positive bacterial infection, in addition to alleviating the symptoms associated with the elevated level of the Gram-positive bacteria.

In some embodiments, the compositions of the invention are for use in the treatment or prevention of infections of Gram-positive bacteria of the genus selected from the list consisting of: Clostridium, Staphylococcus, Enterococcus spp, Bacillus, Erysipelothrix or Listeria. In some embodiments, the Gram-positive bacterial infection for treatment or prevention is Clostridium difficile infection. In some embodiments, the Gram-positive bacterial infection for treatment or prevention is Bacillus anthracis infection. In some embodiments, the Gram-positive bacterial infection for treatment or prevention is Clostridium perfringens infection. In some embodiments, the Gram-positive bacterial infection for treatment or prevention is Listeria infection. In some embodiments, the Gram-positive bacterial infection for treatment or prevention is Staphylococcus aureus infection.

In some embodiments, the bacteria that produces valeric acid may be useful in the treatment or prevention of Gram-positive bacterial infections in the gastrointestinal tract of a subject.

In some embodiments, the compositions of the invention are particularly advantageous as they may eliminate or reduce the need to administer to a subject an antibiotic for use in the treatment of the Gram-positive bacterial infection. In some embodiments, the composition of the invention may eliminate or reduce the need to administer to a subject a broad-spectrum antibiotic. These embodiments are particularly advantageous as they treat the infection and prevent the onset of adverse side effects associated with antibiotic therapy, which may occur due to the non-specific targeting of commensal bacteria. In other words, the administration of the antibiotics may lead to the killing of non-pathogenic bacteria. Such non-specific targeting can cause side effects detrimental to the subject undergoing therapy. The compositions of the invention may therefore be effective is reducing these side effects, by reducing the need to administer antibiotics to a subject during the course of treating a Gram-positive bacterial infection.

In some embodiments, the compositions of the invention are for use in the treatment or prevention of C. difficile infection. Valeric acid has been shown to reduce the viability of C. difficile [19]. Therefore, the compositions of the invention may reduce the viability of C. difficile in a subject during the treatment or prevention of C. difficile infection. C. difficile and Bacillus subtilis, as tested in the examples, are both spore-forming Gram-positive bacteria. In some embodiments, the treatment or prevention of the bacterial infection may be achieved without the off-target effects (for example, the killing of non-pathogenic bacteria in the microbiome) associated with traditional therapies such as antibiotic therapies. Reducing the viability of C. difficile without killing other non-pathogenic bacteria is particularly advantageous as it may reduce the recurrence of C. difficile infection in a subject that may occur when other non-pathogenic bacteria are killed during use of therapies of the art. Therefore, in some embodiments, the compositions of the invention are for use in the treatment or prevention of recurrent C. difficile infection.

### EMBODIMENTS OF THE INVENTION

1. A composition comprising a strain of a bacteria for use in the treatment or prevention of a Gram-positive bacterial infection in a subject, wherein the strain produces valeric acid.
2. The composition for use according to embodiment 1, wherein the Gram-positive bacterial infection is in the gastrointestinal tract.
3. The composition for use according to any preceding embodiment, wherein the Gram-positive bacterial infection is a pathogenic Gram-positive bacterial infection.
4. The composition for use according to any preceding embodiment, wherein, when administered to the subject, the composition reduces the viability of the Gram-positive bacteria.
5. The composition for use according to any preceding embodiment, wherein, when administered to the subject, the composition inhibits the growth of the Gram-positive bacteria.
6. The composition for use according to any preceding embodiment, wherein the composition delays the onset of a recurrent infection.
7. The composition for use according to any preceding embodiment, wherein the composition prevents a recurrent infection.
8. The composition for use according to any preceding embodiment, wherein the subject is at risk of developing a Gram-positive bacterial infection.
9. The composition for use according to any preceding embodiment, wherein the subject is an asymptomatic carrier of the Gram-positive bacteria.
10. The composition for use according to any of embodiments 1 to 9, wherein the subject has been administered or is being administered with one or more antibiotics, wherein optionally the one or more antibiotics comprises a broad-spectrum antibiotic.
11. The composition for use according to embodiment 10, wherein the antibiotic is selected from the list consisting of: Vancomycin, Bactrium, Doxycyline, Ceftobiprole, Ceftaroline, Clindamycin, Dalbavancin, Daptomycin, Fusidic acid, Linezolid, Mupirocin, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Linezolid, Streptogramins, Tigecycline and Daptomycin.
12. The composition for use according to any of embodiments 1 to 9, wherein the subject is not to be simultaneously, separately or sequentially administered an antibiotic as part of the same treatment regimen for the treatment or prevention of the Gram-positive bacterial infection.
13. The composition for use according to any preceding embodiment, wherein the Gram-positive bacteria is resistant to antibiotic therapy.
14. The composition for use according to any preceding embodiment, wherein the Gram-positive bacteria is of the genus selected from the list consisting of: Clostridium, Staphylococcus, Enterococcus spp, Bacillus, Erysipelothrix or Listeria.
15. The composition for use according to any preceding embodiment, wherein the Gram-positive bacterial infection is Clostridium difficile infection.
16. The composition for use according to embodiment 15, wherein the composition prevents or treats one or more of the following conditions associated with C. difficile infection: diarrhoea, abdominal pain, pyrexia, haematochezia, dehydration, weight loss, toxic megacolon, gastrointestinal perforation, abdominal distension, colonic distension, nausea, pseudomembranous colitis, multiple organ dysfunction syndrome or sepsis.
17. The composition for use according to any preceding embodiment, wherein the Gram-positive bacterial infection is Bacillus anthracis infection.
18. The composition for use according to any preceding embodiment, wherein the Gram-positive bacterial infection is Clostridium perfringens infection.
19. The composition for use according to any preceding embodiment, wherein the Gram-positive bacterial infection is Listeria infection.
20. The composition for use according to any preceding embodiment, wherein the Gram-positive bacterial infection is Listeria monocytogenes infection.
21. The composition for use according to any preceding embodiment, wherein the Gram-positive bacterial infection is Staphylococcus aureus infection.
22. The composition for use according to any preceding embodiment, wherein the strain is of the genus Megasphaera.
23. The composition for use according to any preceding embodiment, wherein the bacterial strain has a 16S rRNA sequence that is at least 95%, 96%, 97%, 98%, 99%, 99.5% or 99.9% identical to the 16S rRNA sequence of a bacterial strain of the genus Megasphaera.\
24. The composition for use according to any preceding embodiment, wherein the bacterial strain has a 16s rRNA gene sequence that is at least 95%, 96%, 97%, 98%, 99%, 99.5% or 99.9% identical to any one of SEQ ID NOs:3, 4, 5, 6 or 7 or wherein the bacterial strain has a 16s rRNA gene sequence represented by any one of SEQ ID NOs: 3, 4, 5, 6 or 7.
25. The composition for use according to any preceding embodiment, wherein the strain is of the species Megasphaera massiliensis.
26. The composition for use according to any preceding embodiment, wherein the bacterial strain has a 16s rRNA sequence that is at least 95%, 96%, 97%, 98%, 99%, 99.5% or 99.9% identical to SEQ ID NO:1 or 2.
27. The composition for use according to any preceding embodiment, wherein the bacterial strain has a 16s rRNA sequence that is at least 95%, 96%, 97%, 98%, 99%, 99.5% or 99.9% identical to SEQ ID NO: 2
28. The composition for use according to any preceding embodiment, wherein the bacteria is the strain deposited at NCIMB under accession number NCIMB 42787.
29. The composition for use according to any preceding embodiment, wherein the bacterial strain is engineered to produce valeric acid.
30. The composition for use according to embodiment 22, wherein the composition does not contain bacteria from any other genus or which comprises only de minimis or biologically irrelevant amounts of bacteria from another genus.
31. The composition for use according to any preceding embodiment, wherein the bacterial strain also produces one or both of butyrate and hexanoic acid.
32. The composition for use according to any preceding embodiment, wherein the bacterial strain consumes one or both of acetate and propionate.
33. The composition for use according to any preceding embodiment, wherein the bacterial strain also produces butyrate and hexanoic acid and consumes acetate and propionate.
34. The composition of any preceding embodiment, wherein the composition is for oral administration.
35. The composition of any preceding embodiment, wherein the composition comprises one or more pharmaceutically acceptable excipients or carriers.
36. The composition of any preceding embodiment, wherein the bacterial strain is lyophilised.
37. The composition of any preceding embodiment, wherein the bacterial strain is viable and capable of partially or totally colonising the intestine.
38. The composition of any preceding embodiment, wherein the composition comprises a single strain of the genus Megasphaera.
39. The composition of any preceding embodiment, which comprises the Megasphaera bacterial strain as part of a microbial consortium.
40. A food product comprising the composition of any preceding embodiment, for the use of any preceding embodiment.
41. An antibiotic selected from the list consisting of: Vancomycin, Bactrium, Doxycyline, Ceftobiprole, Ceftaroline, Clindamycin, Dalbavancin, Daptomycin, Fusidic acid, Linezolid, Mupirocin, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Linezolid, Streptogramins, Tigecycline and Daptomycin, for use in the treatment of a Gram-positive bacterial infection in a subject, wherein the subject is to be administered the composition of any of embodiments 1 to 36.
42. A combination comprising a composition according to any of embodiments 1 to 36 and an antibiotic selected from the list consisting of: Vancomycin, Bactrium, Doxycyline, Ceftobiprole, Ceftaroline, Clindamycin, Dalbavancin, Daptomycin, Fusidic acid, Linezolid, Mupirocin, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Linezolid, Streptogramins, Tigecycline and Daptomycin, for use in the treatment or prevention of a Gram-positive bacterial infection in a subject.
43. An antibiotic for use in the treatment of a Gram-positive bacterial infection in a subject, wherein the subject has been administered or is to be administered the composition of any of embodiments 1-36.
44. A cell of the Megasphaera massiliensis strain deposited as NCIMB 42787, or a derivative thereof, for use in a method defined in any of embodiments 1-36.
45. A composition comprising one or more bacterial strain of the species Megasphaera massiliensis, for use in a method defined in any of embodiments)-36.
46. A method of treating or preventing a Gram-positive bacterial infection, comprising administering a composition of any preceding embodiment to a subject in need thereof
47. A bacterial strain for use in therapy, wherein the bacterial strain has a 16S rRNA sequence that is at least 95%, 96%, 97%, 98%, 99%, 99.5% or 99.9% identical to any one of SEQ ID NOs:3, 4, 5, 6 or 7.
48. A bacterial strain having the 16S rRNA sequence represented by any one of SEQ ID NOs: 3, 4, 5, 6 or 7 for use in therapy.

### BRIEF DESCRIPTION OF DRAWINGS

The invention is illustrated by reference to the following drawings, in which:

FIG. 1 shows an example of the Gram-positive bacterial infection model assay

FIG. 2 shows that strain 42787 reduces outgrowth of B. subtilis

FIG. 3 shows a second replicate of the Strain 42787 inhibiting the outgrowth of B. subtilis

FIG. 4 shows that strain 42787 produces valeric acid

FIG. 5 shows the levels of metabolite production—valeric acid in the supernatant

FIG. 6 shows the levels of metabolite production organic acids in the supernatant.

FIG. 7: Organic acid production and consumption by NCIMB 42787, NCIMB 43385, NCIMB 43388 and NCIMB 43389.

FIGS. 8A-8B: Administration of NCIMB 42787 significantly reduces levels of TNFα produced by mice splenocytes after ConA stimulation ex vivo (FIG. 8A—unstimulated; FIG. 8B—ConA stimulated).

FIGS. 9A-9B: FIG. 9A shows that NCIMB 43385 and NCIMB 43389 supernatants increase levels of occludin (top) and E-cadherin (bottom) in HCT116 cells. FIG. 9B shows that NCIMB 43385 supernatants significantly increase levels of occludin (top) and E-cadherin (bottom) in HT29 cells.

### DISCLOSURE OF THE INVENTION

Bacterial Strains

The compositions of the invention comprise a bacterial strain useful for treating or preventing Gram-positive bacterial infections. The bacterial strain of the invention exhibits the unusual property of producing valeric acid. Valeric acid (also known as pentanoic acid) has been shown to reduce the viability of the Gram-positive pathogenic bacteria C. difficile [19] and the examples show that a strain of M. massiliensis that produces valeric acid is effective for reducing the viability of a Gram-positive bacteria.

The inventors have found that the bacteria of the invention inhibit the growth and/or reduce the viability of Gram-positive bacteria. For example, the inventors have found that particular bacterial strains inhibit the growth and/or reduce the viability of the Gram-positive bacteria Bacillus subtilis in an in vitro Gram-positive bacteria model of infection. Thus, bacterial strains of the invention may be effective for use in the treatment or prevention of Gram-positive bacterial infections. In particular, the compositions of the invention may be useful in the treatment or prevention of pathogenic Gram-positive bacterial infections.

The compositions of the invention comprise a bacterial strain that produces valeric acid. In some embodiments, the bacterial strain of the invention is a commensal bacterial strain. A commensal bacterial strain is a symbiont derived from the gastrointestinal tract of a mammal, such as a human. Examples of genera from which the commensal bacterial strain may be derived include Bacteroides, Enterococcus, Escherichia, Klebsiella, Bifidobacterium, Staphylococcus, Lactobacillus, Megasphaera, Clostridium, Proteus, Pseudomonas, Salmonella, Faecalibacterium, Peptostreptococcus or Peptococcus. In some embodiments, the commensal bacterial strain is of the genus Megasphaera. Preferably, the bacterial strain for use in the invention has a 16S rRNA sequence that is at least 95%, 96%, 97%, 98%, 99%, 99.5% or 99.9% identical to SEQ ID NO:1 or 2. Preferably, the sequence identity is to SEQ ID NO:2. Preferably, the bacterial strain for use in the invention has the 16S rRNA sequence represented by SEQ ID NO:2. Preferably, Megasphaera species for use in the invention include Megasphaera elsdenii, Megasphaera cerevisiae, Megasphaera massiliensis, Megasphaera indica, Megasphaera paucivorans, Megasphaera sueciensis and Megasphaera micronuciformis. A further example of a Megasphaera species for use in the invention is Megasphaera hexanoica. The Megasphaera are obligately anaerobic, lactate-fermenting, gastrointestinal microbe of ruminant and non-ruminant mammals, including humans. Preferably, the bacterial strain is derived from the species to which the composition is intended to be administered. In preferred embodiments of every aspect of the invention, the composition comprises a strain of the species Megasphaera massiliensis.

The type strain of M. massiliensis is NP3 (=CSUR P245=DSM 26228) [20]. The GenBank accession number for the 16S rRNA gene sequences of M massiliensis strain NP3 is JX424772.1 (disclosed herein as SEQ ID NO:1).

The Megasphaera massiliensis bacterium tested in the Examples is referred to herein as strain 42787. A 16S rRNA sequence for the 42787 strain that was tested is provided in SEQ ID NO:2. Preferably, the bacteria for use in the invention is of the species Megasphaera massiliensis, in particular the strain 42787.

Strain 42787 was deposited with the international depositary authority NCIMB, Ltd. (Ferguson Building, Aberdeen, AB21 9YA, Scotland) by 4D Pharma Research Ltd. (Life Sciences Innovation Building, Cornhill Road, Aberdeen, AB25 2ZS, Scotland) on 13 Jul. 2017 and was assigned accession number NCIMB 42787.

Bacterial strains closely related to the strain tested in the examples are also expected to be effective for treating or preventing Gram-positive bacterial infections. In certain embodiments, the bacterial strain for use in the invention has a 16S rRNA sequence that is at least 95%, 96%, 97%, 98%, 99%, 99.5% or 99.9% identical to the 16S rRNA sequence of a bacterial strain of Megasphaera massiliensis. Preferably, the bacterial strain for use in the invention has a 16S rRNA sequence that is at least 95%, 96%, 97%, 98%, 99%, 99.5% or 99.9% identical to SEQ ID NO: 2. Preferably, the bacterial strain for use in the invention has the 16S rRNA sequence represented by SEQ ID NO:2.

Bacterial strains that are biotypes of strains 42787 are also expected to be effective for treating or preventing Gram-positive bacterial infections. A biotype is a closely related strain that has the same or very similar physiological and biochemical characteristics.

Strains that are biotypes of 42787 and that are suitable for use in the invention may be identified by sequencing other nucleotide sequences from strain 42787. For example, substantially the whole genome may be sequenced and a biotype strain for use in the invention may have at least 95%, 96%, 97%, 98%, 99%, 99.5% or 99.9% sequence identity across at least 80% of its whole genome (e.g. across at least 85%, 90%, 95% or 99%, or across its whole genome). Other suitable sequences for use in identifying biotype strains may include hsp60 or repetitive sequences such as BOX, ERIC, (GTG)₅, or REP [21]. Biotype strains may have sequences with at least 95%, 96%, 97%, 98%, 99%, 99.5% or 99.9% sequence identity to the corresponding sequence of the strain 42787.

A bacterial strain useful in the invention is one that produces valeric acid and that reduces the growth of Gram-positive bacteria in accordance with the methods disclosed in the Examples. Such a property is a hitherto unusual means of classifying bacteria in the art. Furthermore, the property of a bacterial strain producing valeric acid is a hitherto unrecognised advantage in the context of the treatment of Gram-positive bacterial infections.

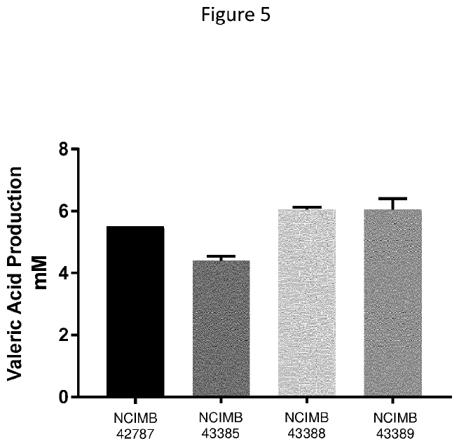

Such bacteria may be identified by routinely profiling the production of metabolites of a bacterial strain. Metabolite profiling using the methods set out in Example 1 can be used to identity candidate bacterial strains that produce valeric acid. The method has identified several other Megasphaera massiliensis strains with the advantageous property of producing valeric acid (see FIG. 5; Strains Ref 1, Ref 2 and Ref 3). The assays disclosed in Example 2 can then be used to verify candidate bacterial strains for use in the treatment or prevention of Gram-positive bacterial infections.

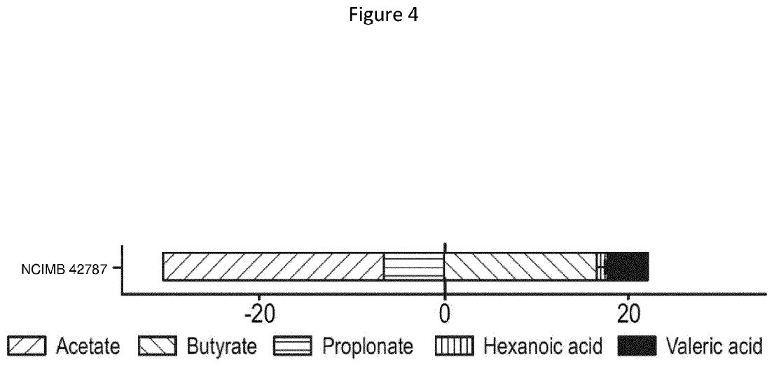

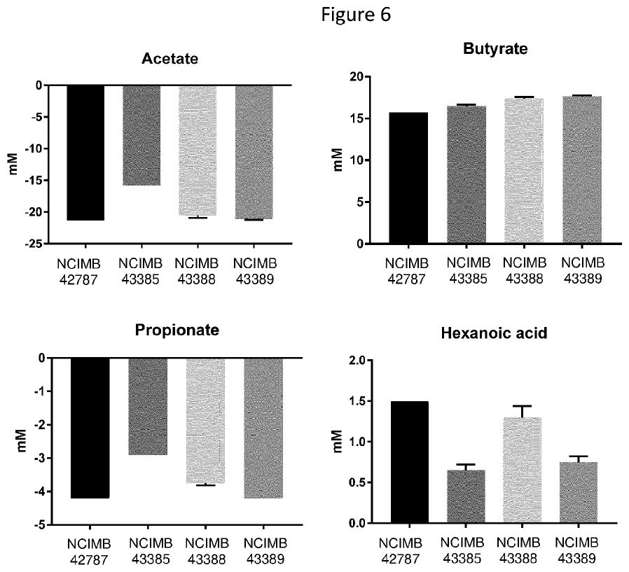

The inventors have found that the bacterial strain used in the Examples produces butyrate and hexanoic acid and consumes acetate and propionate (see FIG. 4). The Megasphaera massiliensis strains Ref 1, Ref 2 and Ref 3 were also found to consume and produce these metabolites (FIGS. 5 and 6). Therefore, in some embodiments, the bacterial strain of the invention also produces one or both of butyrate and hexanoic acid. In some embodiments, the bacterial strain of the invention consumes one or both of acetate and propionate. In preferred embodiments, the bacterial strain of the invention produces butyrate, valeric acid and hexanoic acid and consumes acetate and propionate.

In addition, suitable biotypes capable of producing valeric acid are those that contain metabolic pathways that produce the valeric acid. Such strains can be identified by genomic analysis, for example by determining whether the bacterial strain encodes for enzymes required for the biosynthesis of valeric acid.

Alternatively, strains that are biotypes of strain 42787 and that are suitable for use in the invention may be identified by using strain 42787 and restriction fragment analysis and/or PCR analysis, for example by using fluorescent amplified fragment length polymorphism (FAFLP) and repetitive DNA element (rep)-PCR fingerprinting, or protein profiling, or partial 16S or 23S rDNA sequencing. In preferred embodiments, such techniques may be used to identify other Megasphaera massiliensis strains.

In certain embodiments, strains that are biotypes of strain 42787 and that are suitable for use in the invention are strains that provide the same pattern as strain 42787 when analysed by amplified ribosomal DNA restriction analysis (ARDRA), for example when using Sau3AI restriction enzyme (for exemplary methods and guidance see, for example, [22]). Alternatively, biotype strains are identified as strains that have the same carbohydrate fermentation patterns as strain 42787.

Other bacterial strains that are useful in the compositions and methods of the invention, such as biotypes of 42787, may be identified using any appropriate method or strategy, including the assays described in the examples. In particular, bacterial strains that have similar growth patterns, metabolic type and/or surface antigens to 42787 may be useful in the invention. A useful strain will have comparable immune modulatory activity to 42787. In particular, a biotype strain will elicit comparable effects on the gram-positive bacteria growth reduction assay as shown in the Examples, which may be identified by using the culturing and administration protocols described in the Examples.

A particularly preferred strain of the invention is the Megasphaera massiliensis strain 42787. This is the exemplary strain tested in the examples and shown to be effective for treating or preventing B. subtilis infection in vitro. Therefore, the invention provides a cell, such as an isolated cell, of the Megasphaera massiliensis strain 42787, or a derivative thereof. The invention also provides a composition comprising a cell of the Megasphaera massiliensis strain 42787, or a derivative thereof. The invention also provides a biologically pure culture of the Megasphaera massiliensis strain 42787. The invention also provides a cell of the Megasphaera massiliensis strain 42787, or a derivative thereof, for use in therapy, in particular for the diseases described herein.

A derivative of the strain of the invention may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable therapeutic activity to the 42787 strain. In particular, a derivative strain will elicit comparable effects on the Gram-positive bacterial infection models shown in the Examples, which may be identified by using the culturing and administration protocols described in the Examples. A derivative of the 42787 strain will generally be a biotype of the 42787 strain.

References to cells of the Megasphaera massiliensis 42787 strain encompass any cells that have the same safety and therapeutic efficacy characteristics as the strain 42787, and such cells are encompassed by the invention.

In preferred embodiments, the bacterial strains in the compositions of the invention are viable and capable of partially or totally colonising the intestine.

In certain embodiments, the composition of the invention does not comprise a cell of the Megasphaera massiliensis strain 42787.

In addition, further bacterial strains were deposited with the international depositary authority NCIMB, Ltd. (Ferguson Building, Aberdeen, AB21 9YA, Scotland) by 4D Pharma Research Ltd. (Life Sciences Innovation Building, Cornhill Road, Aberdeen, AB25 2ZS, Scotland) on 6 May 2019 as Megasphaera massiliensis (under accession numbers NCIMB 43388 and NCIMB 43389) and Megasphaera spp. (accession numbers NCIMB 43385, NCIMB 43386 and NCIMB 43387). Accordingly, in an alternative embodiment, the compositions of the invention comprise one or more of these bacterial strains, or biotypes or derivatives thereof. For the avoidance of doubt, Ref 1 referred to above is the strain deposited under accession number NCIMB 43385, Ref 2 referred to above is the strain deposited under accession number NCIMB 43388, and Ref 3 referred to above is the strain deposited under accession number NCIMB 43389.

Bacterial strains closely related to the strains tested in the Examples are also expected to be effective for treating or preventing Gram-positive bacterial infections.

In certain embodiments, the bacterial strain for use in the invention is the Megasphaera massiliensis strain deposited under accession number NCIMB 43388. In certain embodiments, the invention provides a cell of the strain deposited under accession number NCIMB 43388, or a derivative thereof, for use in therapy. In certain embodiments, the invention provides a cell of the strain deposited under accession number NCIMB 43388, or derivative thereof for treating or preventing Gram-positive bacterial infections. In certain embodiments, the invention provides a cell of the strain deposited under accession number NCIMB 43388, for use in any one of the diseases described herein.

In preferred embodiments, the invention provides a composition comprising the strain deposited at NCIMB under accession number NCIMB 43388, or a derivative or biotype thereof, preferably for use in the treatment or prevention of Gram-positive bacterial infections.

In certain embodiments, the composition of the invention does not comprise a cell of the Megasphaera massiliensis strain deposited under accession number NCIMB 43388. In some embodiments, the bacterial strain in the compositions of the invention is a bacterial strain of the genus Megasphaera, wherein the bacterial strain is not the strain deposited under accession number NCIMB 43388. In some embodiments, the bacterial strain in the compositions of the invention is a bacterial strain of the species Megasphaera massiliensis, wherein the bacterial strain is not the strain deposited under accession number NCIMB 43388.

Accordingly, in certain embodiments, the bacterial strain for use in the invention is the Megasphaera massiliensis strain deposited under accession number NCIMB 43389. In certain embodiments, the invention provides a cell of the strain deposited under accession number NCIMB 43389, or a derivative thereof, for use in therapy. In certain embodiments, the invention provides a cell of the strain deposited under accession number NCIMB 43389, or derivative thereof for use in treating or preventing Gram-positive bacterial infections. In certain embodiments, the invention provides a cell of the strain deposited under accession number NCIMB 43389, for use in any one of the diseases described herein.

In preferred embodiments, the invention provides a composition comprising the strain deposited at NCIMB under accession number NCIMB 43389, or a derivative or biotype thereof, preferably for use in the treatment or prevention of Gram-positive bacterial infections.

In certain embodiments, the composition of the invention does not comprise a cell of the Megasphaera massiliensis strain deposited under accession number NCIMB 43389. In some embodiments, the bacterial strain in the compositions of the invention is a bacterial strain of the genus Megasphaera, wherein the bacterial strain is not the strain deposited under accession number NCIMB 43389. In some embodiments, the bacterial strain in the compositions of the invention is a bacterial strain of the species Megasphaera massiliensis, wherein the bacterial strain is not the strain deposited under accession number NCIMB 43389.

In certain embodiments, the bacterial strain for use in the invention has a 16S rRNA sequence that is at least 95%, 96%, 97%, 98%, 99%, 99.5% or 99.9% identical to SEQ ID NO:4. In certain embodiments, the bacterial strain for use in the invention has the 16S rRNA sequence represented by SEQ ID NO:4. In certain embodiments, the invention provides a bacterial strain having a 16S rRNA sequence that is at least 95%, 96%, 97%, 98%, 99%, 99.5% or 99.9% identical to SEQ ID NO:4 for use in therapy. In certain embodiments, the invention provides a bacterial strain having the 16S rRNA sequence represented by SEQ ID NO:4 for use in therapy.

In certain embodiments, the bacterial strain for use in the invention has a 16S rRNA sequence that is at least 95%, 96%, 97%, 98%, 99%, 99.5% or 99.9% identical to SEQ ID NO:5. In certain embodiments, the bacterial strain for use in the invention has the 16S rRNA sequence represented by SEQ ID NO:5. In certain embodiments, the invention provides a bacterial strain having a 16S rRNA sequence that is at least 95%, 96%, 97%, 98%, 99%, 99.5% or 99.9% identical to SEQ ID NO:5 for use in therapy. In certain embodiments, the invention provides a bacterial strain having the 16S rRNA sequence represented by SEQ ID NO:5 for use in therapy.

In certain embodiments, the bacterial strain for use in the invention has a 16S rRNA sequence that is at least 95%, 96%, 97%, 98%, 99%, 99.5% or 99.9% identical to the 16S rRNA sequence of a bacterial strain of the genus Megasphaera. In certain embodiments, the bacterial strain for use in the invention is of the genus Megasphaera.

In certain embodiments, the bacterial strain for use in the invention is the Megasphaera strain deposited under accession number NCIMB 43385. In certain embodiments, the invention provides a cell of the strain deposited under accession number NCIMB 43385, or a derivative thereof, for use in therapy. In certain embodiments, the invention provides a cell of the strain deposited under accession number NCIMB 43385, or derivative thereof for treating or preventing Gram-positive bacterial infections. In certain embodiments, the invention provides a cell of the strain deposited under accession number NCIMB 43385, for use in any one of the diseases described herein.

In preferred embodiments, the invention provides a composition comprising the strain deposited at NCIMB under accession number NCIMB 43385, or a derivative or biotype thereof, preferably for use in the treatment or prevention of Gram-positive bacterial infections.

In certain embodiments, the composition of the invention does not comprise a cell of the Megasphaera massiliensis strain deposited under accession number NCIMB 43385. In some embodiments, the bacterial strain in the compositions of the invention is a bacterial strain of the genus Megasphaera, wherein the bacterial strain is not the strain deposited under accession number NCIMB 43385. In some embodiments, the bacterial strain in the compositions of the invention is a bacterial strain of the species Megasphaera massiliensis, wherein the bacterial strain is not the strain deposited under accession number NCIMB 43385.

In certain embodiments, the bacterial strain for use in the invention is the Megasphaera strain deposited under accession number NCIMB 43386. In certain embodiments, the invention provides a cell of the strain deposited under accession number NCIMB 43386, or a derivative thereof, for use in therapy. In certain embodiments, the invention provides a cell of the strain deposited under accession number NCIMB 43386, or derivative thereof for treating or preventing Gram-positive bacterial infections. In certain embodiments, the invention provides a cell of strain the deposited under accession number NCIMB 43386, for use in any one of the diseases described herein.

In preferred embodiments, the invention provides a composition comprising the strain deposited at NCIMB under accession number NCIMB 43386, or a derivative or biotype thereof, preferably for use in the treatment or prevention of Gram-positive bacterial infections.

In certain embodiments, the composition of the invention does not comprise a cell of the Megasphaera massiliensis strain deposited under accession number NCIMB 43386. In some embodiments, the bacterial strain in the compositions of the invention is a bacterial strain of the genus Megasphaera, wherein the bacterial strain is not the strain deposited under accession number NCIMB 43386. In some embodiments, the bacterial strain in the compositions of the invention is a bacterial strain of the species Megasphaera massiliensis, wherein the bacterial strain is not the strain deposited under accession number NCIMB 43386.

In certain embodiments, the bacterial strain for use in the invention is the Megasphaera strain deposited under accession number NCIMB 43387. In certain embodiments, the invention provides a cell of the strain deposited under accession number NCIMB 43387, or a derivative thereof, for use in therapy. In certain embodiments, the invention provides a cell of the strain deposited under accession number NCIMB 43387, or derivative thereof for treating or preventing Gram-positive bacterial infections. In certain embodiments, the invention provides a cell of strain the deposited under accession number NCIMB 43387, for use in any one of the diseases described herein.

In preferred embodiments, the invention provides a composition comprising the strain deposited at NCIMB under accession number NCIMB 43387, or a derivative or biotype thereof, preferably for use in the treatment or prevention of Gram-positive bacterial infections.

In certain embodiments, the composition of the invention does not comprise a cell of the Megasphaera massiliensis strain deposited under accession number NCIMB 43387. In some embodiments, the bacterial strain in the compositions of the invention is a bacterial strain of the genus Megasphaera, wherein the bacterial strain is not the strain deposited under accession number NCIMB 43387. In some embodiments, the bacterial strain in the compositions of the invention is a bacterial strain of the species Megasphaera massiliensis, wherein the bacterial strain is not the strain deposited under accession number NCIMB 43387.

In certain embodiments, the bacterial strain for use in the invention has a 16S rRNA sequence that is at least 95%, 96%, 97%, 98%, 99%, 99.5% or 99.9% identical to SEQ ID NO:3. In certain embodiments, the bacterial strain for use in the invention has a 16S rRNA sequence that is at least 95%, 96%, 97%, 98%, 99%, 99.5% or 99.9% identical to SEQ ID NO:4. In certain embodiments, the bacterial strain for use in the invention has a 16S rRNA sequence that is at least 95%, 96%, 97%, 98%, 99%, 99.5% or 99.9% identical to SEQ ID NO:6. In certain embodiments, the bacterial strain for use in the invention has a 16S rRNA sequence that is at least 95%, 96%, 97%, 98%, 99%, 99.5% or 99.9% identical to SEQ ID NO:7. In certain embodiments, the bacterial strain for use in the invention has a 16S rRNA sequence that is at least 95%, 96%, 97%, 98%, 99%, 99.5% or 99.9% identical to SEQ ID NOs:3, 4, 6 or 7. In certain embodiments, the invention provides a bacterial strain having a 16S rRNA sequence that is at least 95%, 96%, 97%, 98%, 99%, 99.5% or 99.9% identical to SEQ ID NOs:3, 4, 6 or 7 for use in therapy.

In certain embodiments, the bacterial strain for use in the invention has the 16S rRNA sequence represented by SEQ ID NO:3. In certain embodiments, the bacterial strain for use in the invention has the 16S rRNA sequence represented by SEQ ID NO:4. In certain embodiments, the bacterial strain for use in the invention has the 16S rRNA sequence represented by SEQ ID NO:6. In certain embodiments, the bacterial strain for use in the invention has the 16S rRNA sequence represented by SEQ ID NO:7. In certain embodiments, the bacterial strain for use in the invention has the 16S rRNA sequence represented by SEQ ID NOs:3, 4, 6 or 7. In certain embodiments, the invention provides a bacterial strain having the 16S rRNA sequence represented by SEQ ID NOs: 3, 4, 6 or 7 for use in therapy.

Bacterial strains that are biotypes of one or more of the strains deposited under accession numbers NCIMB 43385, NCIMB 43386, NCIMB 43387, NCIMB 43388 and/or NCIMB 43389 are also expected to be effective for treating or preventing Gram-positive bacterial infections. A biotype is a closely related strain that has the same or very similar physiological and biochemical characteristics.

In certain embodiments, the invention provides the bacterial strains deposited under accession numbers NCIMB 43385, NCIMB 43386, NCIMB 43387, NCIMB 43388 and/or NCIMB 43389, or biotypes thereof, for use in therapy.

Strains that are biotypes of one or more of the strains deposited under accession numbers NCIMB 43385, NCIMB 43386, NCIMB 43387, NCIMB 43388 and/or NCIMB 43389 and that are suitable for use in the invention may be identified by sequencing other nucleotide sequences for one or more of the strains deposited under accession numbers NCIMB 43385, NCIMB 43386, NCIMB 43387, NCIMB 43388 and/or NCIMB 43389. For example, substantially the whole genome may be sequenced and a biotype strain for use in the invention may have at least 85%, 90%, 95%, 96%, 97%, 98%, 99%, 99.5% or 99.9% sequence identity across at least 80% of its whole genome (e.g. across at least 85%, 90%, 95% or 99%, or across its whole genome). Other suitable sequences for use in identifying biotype strains may include hsp60 or repetitive sequences such as BOX, ERIC, (GTG)₅, or REP. Biotype strains may have sequences with at least 85%, 90%, 95%, 96%, 97%, 98%, 99%, 99.5% or 99.9% sequence identity to the corresponding sequence of one or more of the strains deposited under accession numbers NCIMB 43385, NCIMB 43386, NCIMB 43387, NCIMB 43388 and/or NCIMB 43389.

Alternatively, strains that are biotypes of one or more of the strains deposited under accession numbers NCIMB 43385, NCIMB 43386, NCIMB 43387, NCIMB 43388 and/or NCIMB 43389 and that are suitable for use in the invention may be identified by using one or more of the strains deposited under accession numbers NCIMB 43385, NCIMB 43386, NCIMB 43387, NCIMB 43388 and/or NCIMB 43389 and restriction fragment analysis and/or PCR analysis, for example by using fluorescent amplified fragment length polymorphism (FAFLP) and repetitive DNA element (rep)-PCR fingerprinting, or protein profiling, or partial 16S or 23 S rDNA sequencing. In preferred embodiments, such techniques may be used to identify other Megasphaera massiliensis strains.

In certain embodiments, strains that are biotypes of one or more of the strains deposited under accession numbers NCIMB 43385, NCIMB 43386, NCIMB 43387, NCIMB 43388 and/or NCIMB 43389 and that are suitable for use in the invention are strains that provide the same pattern as one or more of the strains deposited under accession numbers NCIMB 43385, NCIMB 43386, NCIMB 43387, NCIMB 43388 and/or NCIMB 43389 when analysed by amplified ribosomal DNA restriction analysis (ARDRA), for example when using Sau3AI restriction enzyme. Alternatively, biotype strains are identified as strains that have the same carbohydrate fermentation patterns as one or more of the strains deposited under accession numbers NCIMB 43385, NCIMB 43386, NCIMB 43387, NCIMB 43388 and/or NCIMB 43389.

Other strains that are useful in the compositions and methods of the invention, such as biotypes of one or more of the strains deposited under accession numbers NCIMB 43385, NCIMB 43386, NCIMB 43387, NCIMB 43388 and/or NCIMB 43389, may be identified using any appropriate method or strategy, including the assays described in the Examples.

In certain embodiments, preferred strains of the invention are the strains deposited under accession numbers NCIMB 43385, NCIMB 43386, NCIMB 43387, NCIMB 43388 and/or NCIMB 43389. Therefore, the invention provides a cell, such as an isolated cell, of one or more of the strains deposited under accession numbers NCIMB 43385, NCIMB 43386, NCIMB 43387, NCIMB 43388 and/or NCIMB 43389, or a derivative thereof. The invention also provides a composition comprising a cell of one of more of the strains deposited under accession numbers NCIMB 43385, NCIMB 43386, NCIMB 43387, NCIMB 43388 and/or NCIMB 43389, or a derivative thereof. The invention also provides a biologically pure culture of one or more of the strains deposited under accession numbers NCIMB 43385, NCIMB 43386, NCIMB 43387, NCIMB 43388 and/or NCIMB 43389. The invention also provides a cell of one or more of the strains deposited under accession numbers NCIMB 43385, NCIMB 43386, NCIMB 43387, NCIMB 43388 and/or NCIMB 43389, or a derivative thereof, for use in therapy, in particular for the diseases described herein.

A derivative of the strain of the invention may be a daughter strain (progeny) or a strain cultured (subcloned) from the original. A derivative of a strain of the invention may be modified, for example at the genetic level, without ablating the biological activity. In particular, a derivative strain of the invention is therapeutically active. A derivative strain will have comparable therapeutic activity to one or more of the strains deposited under accession numbers NCIMB 43385, NCIMB 43386, NCIMB 43387, NCIMB 43388 and/or NCIMB 43389. In particular, a derivative strain will produce valeric acid elicit comparable effects on the Gram-positive bacterial infection models shown in the Examples, which may be identified by using the culturing and administration protocols described in the Examples. A derivative of one or more of the strains deposited under accession numbers NCIMB 43385, NCIMB 43386, NCIMB 43387, NCIMB 43388 and/or NCIMB 43389 will generally be a biotype of one or more of the strains deposited under accession numbers NCIMB 43385, NCIMB 43386, NCIMB 43387, NCIMB 43388 and/or NCIMB 43389, respectively.

References to cells of one or more of the strains deposited under accession numbers NCIMB 43385, NCIMB 43386, NCIMB 43387, NCIMB 43388 and/or NCIMB 43389 encompass any cells that have the same safety and therapeutic efficacy characteristics as one or more of the strains deposited under accession numbers NCIMB 43385, NCIMB 43386, NCIMB 43387, NCIMB 43388 and/or NCIMB 43389, respectively, and such cells are encompassed by the invention.

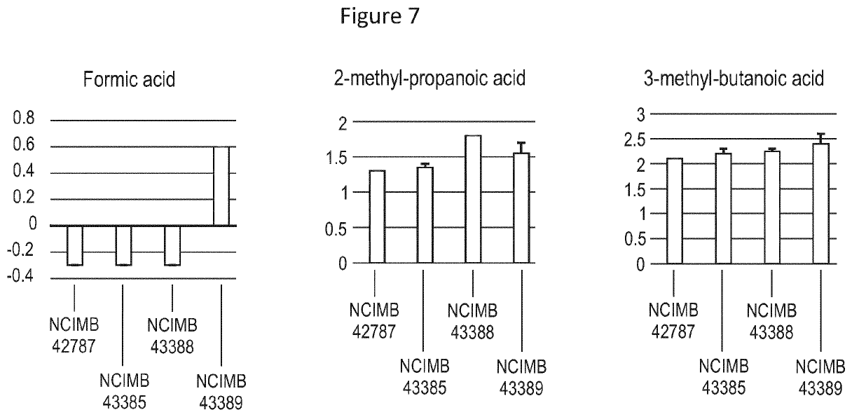

The inventors have found that the bacterial strain used in the Examples produces 2-methyl-propanoic acid and 3-methyl-propanoic acid and consumes formic acid (see FIG. 7). The strains deposited under accession numbers NCIMB 43385, NCIMB 43388 and NCIMB 43389 were also found to produce 2-methyl-propanoic acid and 3-methyl-propanoic acid. In addition, strains deposited under accession numbers NCIMB 43385 and NCIMB 43388 were also found to consume formic acid. Therefore, in some embodiments, the bacterial strain of the invention produces one or more of the metabolites 2-methyl-propanoic acid and 3-methyl-propanoic acid. In some embodiments, the bacterial strain of the invention consumes formic acid. In some embodiments, the bacterial strain of the invention produces 2-methyl-propanoic acid and 3-methyl-propanoic acid and consumes formic acid. In preferred embodiments, the bacterial strain of the invention produces butyrate, valeric acid, hexanoic acid, 2-methyl-propanoic acid and 3-methyl-propanoic acid, and consumes acetate, propionate and formic acid.

In certain embodiments, the production of SCFAs, for example, valeric acid by a strain of the invention or by a strain for use in the invention can be determined using gas chromatography/mass spectrometry (GC/MS). In certain embodiments, gas chromatography/mass spectrometry is used to analyse the content of SCFAs, for example valeric acid, in the cell-free supernatant isolated after culturing the bacterial strain of the invention or for use in the invention. In certain embodiments, the samples of SCFAs in the cell-free supernatants are acidified using hydrochloride acid before GC/MS analysis. In certain exemplified embodiments, the GS/MS analysis is performed using a high polarity column installed in a gas chromatograph coupled with a quadropole detector. In certain embodiments, deuterium labelled internal standards are added prior to GS/MS analysis. In a preferred embodiment, the strain of the invention or strain for use in the invention produces valeric acid, wherein the production of valeric acid is determined using gas chromatography/mass spectrometry (GC/MS) analysis. Gas chromatography/mass spectrometry (GC/MS) is a technique routine to the skilled person, in particular for the detection and analysis of small molecules.

In certain embodiments, the Megasphaera species for use in the invention include Megasphaera cerevisiae, Megasphaera massiliensis, Megasphaera indica, Megasphaera paucivorans, Megasphaera sueciensis, Megasphaera micronuciformis and Megasphaera hexanoica. In certain embodiments, the bacterial strain for use in the invention is of the species Megasphaera cerevisiae, Megasphaera massiliensis, Megasphaera indica, Megasphaera paucivorans, Megasphaera sueciensis, Megasphaera micronuciformis or Megasphaera hexanoica. In certain embodiments, the bacterial strain for use in the invention has a 16S rRNA gene sequence that is at least 95%, 96%, 97%, 98%, 99%, 99.5% or 99.9% identical to the 16S rRNA gene sequence of a bacterial strain of the species Megasphaera cerevisiae, Megasphaera massiliensis, Megasphaera indica, Megasphaera paucivorans, Megasphaera sueciensis, Megasphaera micronuciformis or Megasphaera hexanoica.

In certain embodiments, the compositions of the invention do not comprise Megasphaera elsdenii. In certain embodiments, the bacterial strain useful in the compositions and methods of the invention is not Megasphaera elsdenii.

In certain embodiments, the bacterial strain of the invention is isolated from a human. In certain embodiments, the bacterial strain for use in the invention is isolated from a human.

Therapeutic Uses

As demonstrated in the examples, the bacterial compositions of the invention are effective in treating or preventing Gram-positive bacterial infections. In particular, treatment with the compositions of the invention reduce the viability and/or inhibit the growth of Gram-positive bacteria. Elevated levels of pathogenic Gram-positive bacteria are associated with infection. Therefore, the composition of the invention is for use in the treatment or prevention of a pathogenic Gram-positive bacterial infection. A prominent pathogenic Gram-positive bacteria is Clostridium difficile. Therefore, in some embodiments, the composition of the invention is for use in the treatment or prevention of C. difficile infection (CDI). In some embodiments, the composition of the invention is for use in the treatment of and/or prevention of symptoms associated with CDI.

The inventors have also identified that the bacteria of the invention may be used to prevent a Gram-positive bacterial infection in a subject at risk of a Gram-positive bacterial infection. The compositions of the invention may be administered to an “at-risk” subject such that the probability of the subject developing a Gram-positive bacterial infection is reduced. The compositions of the invention may therefore be useful in the prevention of a Gram-positive bacterial infection.

“At risk” subjects are those who are more susceptible to developing a Gram-positive bacterial infection in comparison to a member of the general public. “At risk” subjects include, but are not limited to, the elderly, immunocompromised, or subjects who are asymptomatic carriers of a pathogenic Gram-positive bacteria. A subgroup susceptible to a particular Gram-positive bacterial infection will vary depending on the type of Gram-positive bacterial infection.

In some embodiments, the composition of the invention may be useful for the prevention or treatment of a Gram-positive bacterial infection in a subject at risk of a Gram-positive bacterial infection. For example, in at-risk elderly or immunocompromised subjects, including those undergoing or who have undergone recently antibiotic therapy, chemotherapy, steroid treatment, or some other such therapy that weakens the immune system, the composition of the invention may be useful in reducing the risk of the subject contracting a Gram-positive bacterial infection, such as C. difficile infection. Therefore, the compositions of the invention may be useful in these subjects in order to treat or prevent Gram-positive bacterial infections.

In some embodiments, the compositions of the invention are particularly advantageous as they may eliminate or reduce the need to administer to a subject an antibiotic for use in the treatment of the Gram-positive bacterial infection. In some embodiments, the composition of the invention may eliminate or reduce the need to administer to a subject a broad-spectrum antibiotic. These embodiments are particularly advantageous as they treat the infection and prevent the onset of adverse side effects associated with the antibiotic therapy. This is because the antibiotic therapy is not administered to the subject in order to treat the infection. Therefore, in some embodiments, the composition of the invention may forgo the need to treat or prevent Gram-positive bacterial infections by administering antibiotics to the subject. In other words, in some embodiments, the adverse side effects associated with antibiotic therapy are avoided during the treatment of a gram-positive bacterial infection, because antibiotics are not administered to the subject.

In some embodiments, the composition of the invention is for use in the treatment or prevention of a Gram-positive bacterial infection, wherein the subject is not to be or has not been simultaneously, separately, or sequentially administered with an antibiotic. As used herein, “simultaneous” means that the antibiotic and the composition of the invention are to be or have been administered at the same time as part of the same treatment regimen; “sequentially” means that doses of the antibiotic and the composition of the invention are to be or have been administered concurrently as part of the same treatment regimen; and “separately” means that the complete dosage of the antibiotic and the composition of the invention are to be or have been administered one after the after as part of the same treatment regimen. In the context of these embodiments, “treatment regimen” refers to the prescription of a treatment programme comprising the administration of both an antibiotic and the composition of the invention to a subject, i.e. where a physician actively prescribes both of the antibiotic and the composition of the invention at the same time for the treatment or prevention of a Gram-positive bacterial infection. “Treatment regimen” does not cover scenarios where a subject is first prescribed, for example, an antibiotic, and is then subsequently prescribed the composition of the invention in response to a sub-optimal clearance of the Gram-positive bacterial infection by the antibiotic.

In some embodiments, the composition is for use in a subject who has not responded to antibiotic therapy. Thus, the composition is particularly advantageous for the treatment of antibiotic-resistant Gram-positive bacterial infections in a subject.

In some embodiments, the compositions of the invention may be used to eliminate asymptomatic levels of Gram-positive bacteria, where “asymptomatic” refers to a level of the Gram-positive bacteria present in a subject that is below the concentration threshold required to cause infection. Therefore, the compositions of the invention may be for use in the prevention of Gram-positive bacterial infection, as they prevent the potential emergence of infection in asymptomatic carriers of pathogenic Gram-positive bacteria.

In some embodiments, the composition of the invention is for use in a subject who has been administered or is being administered with one or more antibiotics. The one or more antibiotics may comprise a broad-spectrum antibiotic. A “broad-spectrum antibiotic” is an antibiotic that targets multiple disease-causing bacteria. Such antibiotics are used, for example, when multiple bacterial infections are present or when a bacterial infection is suspected but the identity of the pathogenic bacteria is unknown. Broad-spectrum antibiotics are effective but they target a wide array of bacteria and may therefore also eliminate or reduce non-pathogenic bacteria. Other antibiotics that are not broad-spectrum may still eliminate or reduce the levels of non-pathogenic bacteria in the microbiome. Therefore, the composition of the invention is particularly advantageous when used in combination with antibiotics, as it repopulates the gut in a subject with a non-pathogenic bacteria during or after administration of the antibiotic. In some embodiments, the bacterial strain of the composition is resistant to an antibiotic that has been or is being administered to the subject. This is advantageous as it means that the antibiotic and the composition of the invention can be administered in combination without the antibiotic inhibiting the therapeutic activity of the bacterial strain. In some embodiments, the composition of the invention is engineered to acquire resistance to an antibiotic that is being administered or is to be administered to the subject.

In some embodiments the compositions of the invention are administered as a combination therapy comprising one or more antibiotics. In some embodiments, the combination therapy may reduce the administration duration of the one or more antibiotics. Without wishing to be bound by theory, the composition of the invention may reduce the administration period for antibiotics or reduce the recurrence of infection following treatment with antibiotics by eliminating the residual pool of pathogenic Gram-positive bacteria that may remain at the end of an antibiotic treatment phase, thereby reducing or preventing the recurrence or re-emergence of the Gram-positive bacterial infection. In some embodiments, the strain of the composition of the invention is resistant to the antibiotic of the combination therapy. This is advantageous because it allows the overlap (i.e. the period in which both elements of the combination therapy are administered to the subject) to be greater, as the strain of the composition is viable in the presence of the antibiotic.

As used herein, the terms “the combination of the invention”, “the therapeutic combination of the invention” and “the therapeutic combination” may be used interchangeably and refer to a therapeutic combination of: (a) a composition comprising a bacterial strain of the invention; and (b) an antibiotic as disclosed herein. It is to be understood that the term “combination” in the context of the therapeutic combination does not refer to components (a) and (b) of the combination necessarily being in the same composition and/or administered at the same time.

In some embodiments, the compositions of the invention may be administered to a subject who has been administered or is being administered with one or more antibiotics selected from the list consisting of: Vancomycin, Bactrium, Doxycyline, Ceftobiprole, Ceftaroline, Clindamycin, Dalbavancin, Daptomycin, Fusidic acid, Linezolid, Mupirocin, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Linezolid, Streptogramins, Tigecycline and Daptomycin.

Preferably, the composition of the invention is resistant to the one or more antibiotics, which allows a greater overlap in the period of administration of the one or more antibiotics and the composition of the invention as a therapeutic combination.

Treatment or prevention may refer to, for example, an alleviation of the severity of symptoms or a reduction in the frequency of exacerbations or the range of triggers that are a problem for the patient.

Clostridium difficile Infection (CDI)

Clostridium difficile (also referred to as C. difficile or C diff) infection refers to a collection of pathological symptoms manifest when the gastrointestinal tract of a subject is colonised with C cliff CDI is often referred to as C diff associated disease (CDAD).

CDI typically occurs when C. difficile spores enter and subsequently colonise the gastrointestinal tract (also known as the “gut”) of a subject. The propensity of C diff spores to colonise the gut is dependent on the microbiomic and metabolomic profile of the subject. For this reason, subjects treated with antibiotics that eradicate the commensal microbiome are more at risk of developing CDI, since the elimination (even if temporary) of the commensal microbiome provides an environment in which C diff colonisation can thrive. Other “at risk” subject groups (i.e. those more prone to the development of CDI) include the elderly (subjects at least 65 years old), subjects with nutritionally imbalanced diets or immunocompromised individuals, such as those receiving or having received immunotherapies, chemotherapies or radiation therapies or those who are undergoing or who have undergone a course of steroid treatment. CDI may also manifest spontaneously, such as in subjects carrying asymptomatic levels of C. diff. The compositions of the invention may be for use in the treatment or prevention of CDI in any subject group.

CDI refers to a collection of symptoms ranging from mild diarrhoea to toxic megacolon or severe colitis. For example, subjects with CDI may be suffering from one or more conditions selected from the list consisting of: diarrhoea, abdominal pain, pyrexia, haematochezia, dehydration, weight loss, toxic megacolon, gastrointestinal perforation, abdominal distension, colonic distension, nausea, pseudomembranous colitis, multiple organ dysfunction syndrome or sepsis. This non-exhaustive list is collectively referred to herein as “the symptoms associated with CDI”. The composition of the invention may be for use in the treatment or prevention of any one or a combination of these symptoms associated with CDI.

In some embodiments, the composition of the invention is for use in the treatment or prevention of one or more of the symptoms associated with CDI in a subject diagnosed with elevated levels of C. diff.

In some embodiments, the composition of the invention is for use in reducing the levels of C. diff in a subject in the treatment or prevention of one or more of the symptoms associated with CDI.

CDI is contractible and can be spread when a group of at-risk subjects spend time in close proximity. For example, the incidence of CDI is greater in subjects resident in nursing homes and in hospitalised subjects. As used herein, “greater” refers to the level of incidence in any subject subgroup relative to the level of incidence in the general population. The composition of the invention may therefore be advantageously administered to subjects at-risk of CDI in order to prevent CDI or to prevent the development of one or more of the symptoms associated CDI.

In some embodiments, the composition of the invention is for use in preventing levels of C. diff increasing in a subject to prevent the onset of one or more symptoms associated with CDI in the subject.

In some embodiments, the compositions of the invention may be for administration to a subject diagnosed with CDI. CDI can be diagnosed using diagnostic kits that detect the level of one or more C. diff toxins in a sample from a subject. Diagnosis may be pre-symptomatic (i.e. it is possible to diagnose asymptomatic carriers of C. diff), meaning that the composition of the invention can be administered to a subject before the emergence of one or more symptoms associated with CDI.

The detection of C. diff toxins (e.g. Toxin A and/or Toxin B) in the blood plasma of a subject is indicative of CDI. Other diagnostic tests can be used, such as nucleic acid amplification of gene sequences specific to C. cliff (e.g. the 16s RNA sequence of C. diff), the detection of products of C. cliff (e.g. glutamate dehydrogenase (GDH), Aromatic fatty acids, TcdA and/or TcdB) and/or toxigenic culture. Not all of the above methods may differentiate between “asymptomatic carriers” of C. diff and those with CDI, however, any of the above diagnostic methods may be used to determine whether a subject is at least an asymptomatic carrier of C. cliff Subjects diagnosed as at least asymptomatic carriers of C. diff may be suitable for administration of the composition of the invention in the treatment or prevention of CDI. The use of the compositions in subjects diagnosed as asymptomatic carriers, as well as those in “at risk” subject groups, advantageously reduces the level of C. diff in a subject before the emergence of symptoms associated with CDI, thereby avoiding the need for additional treatments for one or more of the symptoms associated with CDI.

The recurrent incidence of CDI is high. In other words, once a subject has suffered from CDI, or an initial episode of CDI has been treated, the chance of recurrence in around 8 weeks is 15-25%. For subjects who have suffered a recurrence, the likelihood of further recurrence is 60-80%.

In some embodiments, the composition of the invention is for use in the prevention of recurrent CDI. In other words, the compositions of the invention may be for use in delaying or preventing the recurrence CDI. Without wishing to be bound by theory the compositions of the invention may be particularly beneficial for preventing recurrent infections by inhibiting growth of asymptomatic levels of C. diff remaining after infection to allow the normal microbiota of the GI tract to re-establish itself. In some embodiments, the composition of the invention is for use in a subject to reduce the level of C. diff in a subject to prevent recurrent CDI. In some embodiments, the composition of the invention is for use in a subject to reduce the level of C. diff in a subject to delay recurrent CDI.

In some embodiments, the level of C. diff is reduced in stool in the subject following administration of a composition of the invention. In some embodiments, the level of C. diff is reduced in a stool sample from the subject. In some embodiments, the level of C. diff is reduced in the distal gut of the subject. In some embodiments, the level of C. diff is reduced in the caecum. In some embodiments, the level of C. diff is reduced in the colon. In some embodiments, the level of C. diff is reduced in the rectum. In some embodiments, the level of C. diff is reduced in the small intestine.

In a preferred embodiment, the composition for use in the treatment or prevention of C. difficile infection in a subject comprises a strain of the species Megasphaera massiliensis.

Bacillus anthracis Infection

Bacillus anthracis is a Gram-positive, endospore-forming, rod-shaped bacterium, with a width of 1.0-1.2 μm and a length of 3-5 μm [23]. Elevated levels of B. anthracis are associated with anthrax. In some embodiments, the compositions of the invention are for use in the reduction of levels of B. anthracis in the gastrointestinal tract of a subject for the treatment or prevention of anthrax.

Anthrax occurs in the gastrointestinal tract when ingested spores colonise the gut of a subject. Anthrax in the gastrointestinal tract is rare but is fatal in approximately 25% of cases. The compositions of the invention may therefore be for use in reducing the levels of B. anthracis in the treatment of gastrointestinal anthrax. In preferred embodiments, the composition for use in the treatment or prevention of B. anthracis infection in a subject comprises a strain of the species Megasphaera massiliensis.

Symptoms associated with gastrointestinal anthrax include fever and chills, swelling of neck, painful swallowing, hoarseness, nausea and vomiting (especially bloody vomiting), diarrhoea, flushing and red eyes, and swelling of abdomen. Herein this non-exhaustive list of symptoms are referred to as “symptoms associated with gastrointestinal anthrax”.

The composition of the invention may be for use in the treatment or prevention of any one or more symptoms associated with gastrointestinal anthrax.

In some embodiments, the composition of the invention is for use in the treatment or prevention of one or more of the symptoms associated with gastrointestinal anthrax in a subject with elevated levels of B. anthracis in the gastrointestinal tract.

In some embodiments, the composition of the invention is for use in reducing the level of B. anthracis in a subject in the treatment or prevention of one or more symptoms associated with gastrointestinal anthrax.

In some embodiments, the compositions of the invention may be for administration to a subject diagnosed with gastrointestinal anthrax. Anthrax can be diagnosed using culturing techniques to detect for the presence of B. anthracis. B. anthracis grows well on 5% sheep blood agar and in other routine culture media. Polymyxin-lysozyme-EDTA-thallous acetate can be used to isolate B. anthracis from contaminated specimens. Bicarbonate agar can be used as an identification method to induce capsule formation. B. anthracis usually grows within 24 hours of incubation at 35° C., in ambient air (room temperature) or in 5% CO2. If bicarbonate agar is used for identification, then the medium must be incubated in 5% CO2. B. anthracis colonies are medium-large, grey, flat, and irregular with swirling projections. They are not haemolytic on 5% sheep blood agar. B. anthracis are not motile, susceptible to penicillin, and produce a wide zone of lecithinase on egg yolk agar. Confirmatory testing to identify B. anthracis includes gamma bacteriophage testing, indirect hemagglutination, and enzyme-linked immunosorbent assay to detect antibodies [24]. The best confirmatory precipitation test for anthrax is the Ascoli test, a well-known precipitin test used in the serological diagnosis of anthrax.

In some embodiments, the level of B. anthracis is reduced in stool in the subject following administration of a composition of the invention. In some embodiments, the level of B. anthracis is reduced in a stool sample from the subject. In some embodiments, the level of B. anthracis is reduced in the distal gut of the subject. In some embodiments, the level of B. anthracis is reduced in the caecum. In some embodiments, the level of B. anthracis is reduced in the colon. In some embodiments, the level of B. anthracis is reduced in the rectum. In some embodiments, the level of B. anthracis is reduced in the small intestine.

Clostridium perfringens Infection

Clostridium perfringens in a Gram-positive, rod-shaped spore-forming pathogenic bacterium that is ever present in nature and is often found in the gastrointestinal tracts of mammals. C. perfringens is one of the most common causes of food poisoning in humans. C. perfringens food poisoning occurs in the gastrointestinal tract. C. perfringens infection of the gastrointestinal tract leading to food poisoning is referred to herein as “C. perfringens food poisoning”.

In some embodiments, the composition of the invention is for use in the treatment of C. perfringens infection in a subject. In some embodiments, the compositions of the invention are for use in the treatment of C. perfringens food poisoning. Food poisoning due to C. perfringens is often caused by food that is prepared and kept warm for long periods of time before serving. This environment enables C. perfringens to rapidly multiply and produce bacterial toxins that when ingested cause toxicity in the gastrointestinal tract of a subject. The toxins responsible for food poisoning are heat-resistant and can therefore cause toxicity in a subject even if non-viable C. perfringens is ingested. This often happens when contaminated food is re-heated. However, viable C. perfringens may also be ingested, and produce the toxin in situ.

In some embodiments, the composition of the invention is for use in the treatment of C. perfringens food poisoning in a subject, wherein symptoms of C. perfringens food poisoning have subsisted for more than 24 hours.

In some embodiments, the composition of the invention is for use in the treatment of C. perfringens food poisoning in a subject, wherein symptoms of C. perfringens food poisoning have subsisted for more than 48 hours.

In some embodiments, the composition of the invention is for use in the treatment of C. perfringens food poisoning in a subject, wherein symptoms of C. perfringens food poisoning have subsisted for more than 72 hours.

In some embodiments, the composition of the invention is for use in the treatment of C. perfringens food poisoning in a subject, wherein symptoms of C. perfringens food poisoning have subsisted for more than one week.

Symptoms of C. perfringens food poisoning normally pass within 24 hours, however in subjects where symptoms persist for longer than this period of time, this may indicate that viable C. perfringens has been ingested and is producing toxin in situ. In which case, the composition of the invention may be administered to these subjects in order to reduce the viability of C. perfringens in the treatment of C. perfringens food poisoning. In some embodiments, the composition of the invention is to be administered to these subjects to inhibit the growth of C. perfringens in the treatment of C. perfringens food poisoning.

In some embodiments, the composition of the invention is for use in a subject diagnosed as having C. perfringens infection. In some embodiments, the composition of the invention is for use in a subject diagnosed as having C. perfringens food poisoning. C. perfringens infection may be diagnosed by Nagler's reaction, where the suspected organism is cultured on an egg yolk media plate. One side of the plate contains anti-alpha-toxin, while the other side does not. A streak of suspect organism is placed through both sides. An area of turbidity will form around the side that does not have the anti-alpha-toxin, indicating uninhibited lecithinase activity. In addition, laboratories can diagnose the bacteria by determining the number of bacteria in the feces. Within the 48 hours from when the disease began, if the individual has more than 106 spores of the bacteria per gram of stool, then the illness is diagnosed as C. perfringens food poisoning.

In some embodiments, the level of C. perfringens is reduced in stool in the subject following administration of a composition of the invention. In some embodiments, the level of C. perfringens is reduced in a stool sample from the subject. In some embodiments, the level of C. perfringens is reduced in the distal gut of the subject. In some embodiments, the level of C. perfringens is reduced in the caecum. In some embodiments, the level of C. perfringens is reduced in the colon. In some embodiments, the level of C. perfringens is reduced in the rectum. In some embodiments, the level of C. perfringens is reduced in the small intestine.

In some embodiments, the composition of the invention is for use in the treatment or prevention of one or more symptoms of C. perfringens food poisoning. Symptoms of C. perfringens food poisoning include but are not limited to: diarrhoea, nauseas, abdominal pain, dehydration, fever, loss of appetite and weight loss. This non-exhaustive list of symptoms are referred to herein as “symptoms associated with C. perfringens food poisoning”.

The composition of the invention may be for use in the treatment or prevention of one or more symptoms associated with C. perfringens food poisoning.

In some embodiments, the composition of the invention is for use in the treatment or prevention of one or more of the symptoms associated with C. perfringens food poisoning in a subject with elevated levels of C. perfringens in the gastrointestinal tract.

In some embodiments, the composition of the invention is for use in reducing the level of C. perfringens in a subject in the treatment or prevention of one or more symptoms associated with C. perfringens food poisoning.

In preferred embodiments, the composition for use in the treatment or prevention of C. perfringens infection in a subject comprises a strain of the species Megasphaera massiliensis. In preferred embodiments, the composition for use in the treatment or prevention of C. perfringens food poisoning in a subject comprises a strain of the species Megasphaera massiliensis.

Listeria Infection

Listeria infection, or “Listeriosis”, is a bacterial infection most commonly caused by the Gram-positive bacteria Listeria monocytogenes, although it may also be caused by Listeria ivanovii and Listeria grayi. Listeriosis normally occurs when Listeria is ingested through contaminated food products, such as unpasteurised food products or unwashed vegetables grown in contaminated soil. Therefore, in some embodiments, the composition of the invention is for use in the treatment or prevention of Listeria infection.

Listeriosis may occur during pregnancy, where following infection Listeria can proliferate asymptomatically in the vagina and the uterus. Therefore, in some embodiments, the composition of the invention is for use in the treatment of Listeria infection in a pregnant subject, in particular in the treatment of Listeria infection that has not yet spread to the vagina or uterus.

Infection during pregnancy may lead to neonatal Listeria infection (“neonatal Listeriosis”), which may cause pre-term birth, early-onset sepsis and/or late-onset meningitis in a new-born subject. Therefore, in some embodiments, the composition of the invention may be for use in the prevention of neonatal Listeriosis by reducing the level of Listeria in the gastrointestinal tract of a pregnant subject.

Listeriosis may also cause gastroenteritis in a subject. The composition of the invention may therefore be for use in the treatment or prevention of gastroenteritis in a subject with Listeriosis. Subjects with gastroenteritis caused by Listeria infection may exhibit one or more of the following symptoms: fever, muscle aches, gastrointestinal nausea or diarrhoea, headache, stiff neck, confusion, loss of balance, or convulsions. Herein, these symptoms are referred to as “symptoms associated with Listeria infection in the gastrointestinal tract”.

The composition of the invention may be for use in the treatment or prevention of one or more symptoms associated with Listeria infection in the gastrointestinal tract.

In some embodiments, the composition of the invention is for use in the treatment or prevention of one or more of the symptoms associated with Listeria infection in the gastrointestinal tract in a subject with elevated levels of Listeria in the gastrointestinal tract.

In some embodiments, the composition of the invention is for use in reducing the level of Listeria in a subject in the treatment or prevention of one or more symptoms associated with Listeria gastrointestinal infection.

In some embodiments, the Listeria infection is caused by L. monocytogenes.

In some embodiments, the composition of the invention is for use in a subject diagnosed with Listeria infection. In some embodiments, the composition of the invention is for use in a subject diagnosed with elevated levels of Listeria. In some embodiments, the composition is for use in a subject who is asymptomatic. In some embodiments, the composition is for use in as subject who is symptomatic.

Listeriosis is usually diagnosed when a bacterial culture grows Listeria, such as L. monocytogenes, from a body tissue or fluid, such as blood, spinal fluid, or the placenta.

In some embodiments, the level of Listeria is reduced in stool in the subject following administration of a composition of the invention. In some embodiments, the level of Listeria is reduced in a stool sample from the subject. In some embodiments, the level of Listeria is reduced in the distal gut of the subject. In some embodiments, the level of Listeria is reduced in the caecum. In some embodiments, the level of Listeria is reduced in the colon. In some embodiments, the level of Listeria is reduced in the rectum. In some embodiments, the level of Listeria is reduced in the small intestine.

In preferred embodiments, the composition for use in the treatment or prevention of Listeria infection in a subject comprises a strain of the species Megasphaera massiliensis.

Staphylococcus aureus Infection

Staphylococcus aureus infection may be caused when contaminated food or drink is ingested by a subject. S. aureus infection may cause Staphylococcus enteritis, which is an inflammation of the small intestine caused by S. aureus enterotoxin. Therefore, in some embodiments, the composition of the invention is for use in the treatment or prevention of Staphylococcus aureus infection. In some embodiments, the composition of the invention is for use in the treatment or prevention of Staphylococcus enteritis in a subject with S. aureus infection.

Other symptoms of S. aureus infection include nausea, vomiting, abdominal pain, headache, weakness, diarrhea and fever. This non-exhaustive list of symptoms is herein referred to as “symptoms associated with S. aureus infection”.

The composition of the invention may be for use in the treatment or prevention of any one or more symptoms associated with S. aureus infection in a subject with S. aureus infection.

In some embodiments, the composition of the invention is for use in the treatment or prevention of one or more symptoms associated with S. aureus infection in a subject with elevated levels of S. aureus in the gastrointestinal tract.

In some embodiments, the composition of the invention is for use in reducing the level of S. aureus in a subject in the treatment or prevention of one or more symptoms associated with S. aureus infection.

S. aureus infection may be diagnosed by detecting S. aureus in a stool sample from a subject. In some embodiments, the composition of the invention is for use in a subject diagnosed with S. aureus infection.

In some embodiments, the level of S. aureus is reduced in stool in the subject following administration of a composition of the invention. In some embodiments, the level of S. aureus is reduced in a stool sample from the subject. In some embodiments, the level of S. aureus is reduced in the distal gut of the subject. In some embodiments, the level of S. aureus is reduced in the caecum. In some embodiments, the level of S. aureus is reduced in the colon. In some embodiments, the level of S. aureus is reduced in the rectum. In some embodiments, the level of S. aureus is reduced in the small intestine.

In preferred embodiments, the composition for use in the treatment or prevention of S. aureus infection in a subject comprises a strain of the species Megasphaera massiliensis.

Modes of Administration

Preferably, the compositions of the invention are to be administered to the gastrointestinal tract in order to enable delivery to and/or partial or total colonisation of the intestine with the bacterial strain of the invention. Generally, the compositions of the invention are administered orally, but they may be administered rectally, intranasally, or via buccal or sublingual routes.

In certain embodiments, the compositions comprising the bacterial strain of the invention may be administered as a foam, as a spray or a gel.

In certain embodiments, the compositions of the invention may be administered as a suppository, such as a rectal suppository, for example in the form of a theobroma oil (cocoa butter), synthetic hard fat (e.g. suppocire, witepsol), glycero-gelatin, polyethylene glycol, or soap glycerin composition.

In certain embodiments, the composition of the invention is administered to the gastrointestinal tract via a tube, such as a nasogastric tube, orogastric tube, gastric tube, jejunostomy tube (J tube), percutaneous endoscopic gastrostomy (PEG), or a port, such as a chest wall port that provides access to the stomach, jejunum and other suitable access ports.

The compositions of the invention may be administered once, or they may be administered sequentially as part of a treatment regimen. In certain embodiments, the compositions of the invention are to be administered daily.

In certain embodiments of the invention, treatment according to the invention is accompanied by assessment of the patient's gut microbiota. Treatment may be repeated if delivery of and/or partial or total colonisation with the strain of the invention is not achieved such that efficacy is not observed, or treatment may be ceased if delivery and/or partial or total colonisation is successful and efficacy is observed.

In certain embodiments, the composition of the invention may be administered to a pregnant animal, for example a mammal such as a human in order to prevent a Gram-positive bacterial infection developing in her child in utero and/or after it is born.

The compositions of the invention may be administered to a patient that has been diagnosed with a Gram-positive bacterial infection, or that has been identified as being at risk of a developing a Gram-positive bacterial infection, or that has been identified as an asymptomatic carrier of the Gram-positive bacterial infection. The compositions may also be administered as a prophylactic measure to prevent the development of a Gram-positive bacterial infection in a healthy patient.

The compositions of the invention may be administered to a patient that has been identified as having an abnormal gut microbiota. For example, the patient may have reduced or absent colonisation by Megasphaera, and in particular Megasphaera massiliensis.

The compositions comprising the bacteria of the invention may be administered as a food product, such as a nutritional supplement.

In some embodiments, the composition comprising the bacterial strain and an antibiotic composition are to be administered simultaneously, separately or sequentially. Each of the different compositions may be administered by any combination of the modes of administration described herein. Generally, the compositions of the invention are for the treatment of humans, although they may be used to treat animals including monogastric mammals such as poultry, pigs, cats, dogs, horses or rabbits. The compositions of the invention may be useful for enhancing the growth and performance of animals. If administered to animals, oral gavage may be used.

In some embodiments, the composition of the invention is not administered as a faecal microbial transplant composition.

In certain embodiments, the composition of the invention is for administration to humans. In certain embodiments, the composition for use in the invention is for administration to humans.

Compositions

Generally, the composition of the invention comprises bacteria. In preferred embodiments of the invention, the composition is formulated in freeze-dried form. For example, the composition of the invention may comprise granules or gelatin capsules, for example hard gelatin capsules, comprising a bacterial strain of the invention In some embodiments, each of the separate compositions are formulated in a freeze-dried form. General guidance on the formulation of the compositions of the invention can be found for example, in Aulton's Pharmaceutics: The Design and Manufacture of Medicines.

Preferably, the composition of the invention comprises lyophilised bacteria. Lyophilisation of bacteria is a well-established procedure and relevant guidance is available in, for example, references [25], [ ], [27].

Alternatively, the composition of the invention may comprise a live, active bacterial culture.

In some embodiments, the bacterial strain in the composition of the invention has not been inactivated, for example, has not been heat-inactivated. In some embodiments, the bacterial strain in the composition of the invention has not been killed, for example, has not been heat-killed. In some embodiments, the bacterial strain in the composition of the invention has not been attenuated, for example, has not been heat-attenuated. For example, in some embodiments, the bacterial strain in the composition of the invention has not been killed, inactivated and/or attenuated. For example, in some embodiments, the bacterial strain in the composition of the invention is live. For example, in some embodiments, the bacterial strain in the composition of the invention is viable. For example, in some embodiments, the bacterial strain in the composition of the invention is capable of partially or totally colonising the intestine. For example, in some embodiments, the bacterial strain in the composition of the invention is viable and capable of partially or totally colonising the intestine.

In some embodiments, the composition comprises a mixture of live bacterial strains and bacterial strains that have been killed.

In preferred embodiments, the composition of the invention is encapsulated to enable delivery of the bacterial strain to the intestine. Encapsulation protects the composition from degradation until delivery at the target location through, for example, rupturing with chemical or physical stimuli such as pressure, enzymatic activity, or physical disintegration, which may be triggered by changes in pH. Any appropriate encapsulation method may be used. Exemplary encapsulation techniques include entrapment within a porous matrix, attachment or adsorption on solid carrier surfaces, self-aggregation by flocculation or with cross-linking agents, and mechanical containment behind a microporous membrane or a microcapsule. Guidance on encapsulation that may be useful for preparing compositions of the invention is available in, for example, references [28] and [29].

The composition may be administered orally and may be in the form of a tablet, capsule or powder. Encapsulated products are preferred because Megasphaera are anaerobes. Other ingredients (such as vitamin C, for example), may be included as oxygen scavengers and prebiotic substrates to improve the delivery and/or partial or total colonisation and survival in vivo. Alternatively, the probiotic composition of the invention may be administered orally as a food or nutritional product, such as milk or whey based fermented dairy product, or as a pharmaceutical product.

The composition may be formulated as a probiotic.

A composition of the invention includes a therapeutically effective amount of a bacterial strain of the invention. A therapeutically effective amount of a bacterial strain of the invention is sufficient to exert a beneficial effect upon a patient. A therapeutically effective amount of a bacterial strain may be sufficient to result in delivery to and/or partial or total colonisation of the patient's intestine.

A suitable daily dose of the bacteria, for example for an adult human, may be from about 1×10³ to about 1×10¹¹ colony forming units (CFU); for example, from about 1×10⁷ to about 1×10¹⁰ CFU; in another example from about 1×10⁶ to about 1×10¹⁰ CFU; in another example from about 1×10⁷ to about 1×10¹¹ CFU; in another example from about 1×10⁸ to about 1×10¹⁰ CFU; in another example from about 1×10⁸ to about 1×10¹¹ CFU.

In certain embodiments, the dose of the bacteria is at least 10⁹ cells per day, such as at least 10¹⁰, at least 10¹¹, or at least 10¹² cells per day.

In certain embodiments, the composition contains the bacterial strain in an amount of from about 1×10⁶ to about 1×10¹¹ CFU/g, respect to the weight of the composition; for example, from about 1×10⁸ to about 1×10¹⁰ CFU/g. The dose may be, for example, 1 g, 3 g, 5 g, and 10 g.

In certain embodiments, the invention provides the above pharmaceutical composition, wherein the amount of the bacterial strain is from about 1×10³ to about 1×10¹¹ colony forming units per gram with respect to a weight of the composition.

Typically, a probiotic, such as the composition of the invention, is optionally combined with at least one suitable prebiotic compound. A prebiotic compound is usually a non-digestible carbohydrate such as an oligo- or polysaccharide, or a sugar alcohol, which is not degraded or absorbed in the upper digestive tract. Known prebiotics include commercial products such as inulin and transgalacto-oligosaccharides.

In certain embodiments, the probiotic composition of the present invention includes a prebiotic compound in an amount of from about 1 to about 30% by weight, respect to the total weight composition, (e.g. from 5 to 20% by weight). Carbohydrates may be selected from the group consisting of: fructo-oligosaccharides (or FOS), short-chain fructo-oligosaccharides, inulin, isomalt-oligosaccharides, pectins, xylo-oligosaccharides (or XOS), chitosan-oligosaccharides (or COS), beta-glucans, arable gum modified and resistant starches, polydextrose, D-tagatose, acacia fibers, carob, oats, and citrus fibers. In one aspect, the prebiotics are the short-chain fructo-oligosaccharides (for simplicity shown herein below as FOSs-c.c); said FOSs-c.c. are not digestible carbohydrates, generally obtained by the conversion of the beet sugar and including a saccharose molecule to which three glucose molecules are bonded.

The compositions of the invention may comprise pharmaceutically acceptable excipients or carriers. Examples of such suitable excipients may be found in the reference [30]. Acceptable carriers or diluents for therapeutic use are well known in the pharmaceutical art and are described, for example, in reference [31]. Examples of suitable carriers include lactose, starch, glucose, methyl cellulose, magnesium stearate, mannitol, sorbitol and the like. Examples of suitable diluents include ethanol, glycerol and water. The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s). Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol. Examples of suitable lubricants include sodium oleate, sodium stearate, magnesium stearate, sodium benzoate, sodium acetate, sodium chloride and the like. Preservatives, stabilizers, dyes and even flavouring agents may be provided in the pharmaceutical composition. Examples of preservatives include sodium benzoate, sorbic acid and esters of p-hydroxybenzoic acid. Antioxidants and suspending agents may be also used.

The compositions of the invention may be formulated as a food product. For example, a food product may provide nutritional benefit in addition to the therapeutic effect of the invention, such as in a nutritional supplement. Similarly, a food product may be formulated to enhance the taste of the composition of the invention or to make the composition more attractive to consume by being more similar to a common food item, rather than to a pharmaceutical composition. In certain embodiments, the composition of the invention is formulated as a milk-based product. The term “milk-based product” means any liquid or semi-solid milk- or whey-based product having a varying fat content. The milk-based product can be, e.g., cow's milk, goat's milk, sheep's milk, skimmed milk, whole milk, milk recombined from powdered milk and whey without any processing, or a processed product, such as yoghurt, curdled milk, curd, sour milk, sour whole milk, butter milk and other sour milk products. Another important group includes milk beverages, such as whey beverages, fermented milks, condensed milks, infant or baby milks; flavoured milks, ice cream; milk-containing food such as sweets.

In some embodiments, the compositions of the invention comprise one or more bacterial strains of the genus Megasphaera and do not contain bacteria from any other genera, or which comprise only de minimis or biologically irrelevant amounts of bacteria from another genera. Thus, in some embodiments, the invention provides a composition comprising one or more bacterial strains of the genus Megasphaera, which does not contain bacteria from any other genera or which comprises only de minimis or biologically irrelevant amounts of bacteria from another genera, for use in therapy.

In some embodiments, the compositions of the invention comprise one or more bacterial strains of the species Megasphaera massiliensis and do not contain bacteria from any other species, or which comprise only de minimis or biologically irrelevant amounts of bacteria from another species. Thus, in some embodiments, the invention provides a composition comprising one or more bacterial strains of the species Megasphaera massiliensis, which does not contain bacteria from any other species or which comprises only de minimis or biologically irrelevant amounts of bacteria from another species, for use in therapy.

In some embodiments, the compositions of the invention comprise one or more bacterial strains of the species Megasphaera massiliensis and do not contain bacteria from any other Megasphaera species, or which comprise only de minimis or biologically irrelevant amounts of bacteria from another

Megasphaera species. Thus, in some embodiments, the invention provides a composition comprising one or more bacterial strains of the species Megasphaera massiliensis, which does not contain bacteria from any other Megasphaera species or which comprises only de minimis or biologically irrelevant amounts of bacteria from another Megasphaera species, for use in therapy.

In certain embodiments, the compositions of the invention contain a single bacterial strain or species and do not contain any other bacterial strains or species. Such compositions may comprise only de minimis or biologically irrelevant amounts of other bacterial strains or species. Such compositions may be a culture that is substantially free from other species of organism.

In some embodiments, the invention provides a composition comprising a single bacterial strain of the genus Megasphaera, which does not contain bacteria from any other strains or which comprises only de minimis or biologically irrelevant amounts of bacteria from another strain for use in therapy.

In some embodiments, the invention provides a composition comprising a single bacterial strain of the species Megasphaera massiliensis, which does not contain bacteria from any other strains or which comprises only de minimis or biologically irrelevant amounts of bacteria from another strain for use in therapy.

In some embodiments, the compositions of the invention comprise more than one bacterial strain. For example, in some embodiments, the compositions of the invention comprise more than one strain from within the same species (e.g. more than 1, 2, 3, 4, 5, 6, 7, 8, 9, 10, 15, 20, 25, 30, 35, 40 or 45 strains), and, optionally, do not contain bacteria from any other species. In some embodiments, the compositions of the invention comprise less than 50 strains from within the same species (e.g. less than 45, 40, 35, 30, 25, 20, 15, 12, 10, 9, 8, 7, 6, 5, 4 or 3 strains), and, optionally, do not contain bacteria from any other species. In some embodiments, the compositions of the invention comprise 1-40, 1-30, 1-20, 1-19, 1-18, 1-15, 1-10, 1-9, 1-8, 1-7, 1-6, 1-5, 1-4, 1-3, 1-2, 2-50, 2-40, 2-30, 2-20, 2-15, 2-10, 2-5, 6-30, 6-15, 16-25, or 31-50 strains from within the same species and, optionally, do not contain bacteria from any other species. The invention comprises any combination of the foregoing.

In some embodiments, the composition comprises a microbial consortium. For example, in some embodiments, the composition comprises the Megasphaera bacterial strain as part of a microbial consortium. For example, in some embodiments, the Megasphaera bacterial strain is present in combination with one or more (e.g. at least 2, 3, 4, 5, 10, 15 or 20) other bacterial strains from other genera with which it can live symbiotically in vivo in the intestine. For example, in some embodiments, the composition comprises a bacterial strain of Megasphaera the invention in combination with a bacterial strain from a different genus. In some embodiments, the microbial consortium comprises two or more bacterial strains obtained from a faeces sample of a single organism, e.g. a human. In some embodiments, the microbial consortium is not found together in nature. For example, in some embodiments, the microbial consortium comprises bacterial strains obtained from faeces samples of at least two different organisms. In some embodiments, the two different organisms are from the same species, e.g. two different humans. In some embodiments, the two different organisms are an infant human and an adult human. In some embodiments, the two different organisms are a human and a non-human mammal.

In alternative embodiments, compositions of the invention comprise 16, 15, 14, 13, 12, 11, 10, 9, 8, 7, 6, 5 or fewer distinct bacterial species. In certain embodiments, the composition comprises 4 or fewer distinct bacterial species. In certain embodiments, the composition comprises 3 or fewer distinct bacterial species. In certain embodiments, the composition comprises 2 or fewer distinct bacterial species. In certain embodiments, the composition comprises a species of Megasphaera, in particular Megasphaera massiliensis, and no other bacterial species. In preferred embodiments, the compositions of the invention comprise a single strain Megasphaera, in particular a single strain of Megasphaera massiliensis, and no other bacterial strains or species. Such compositions may comprise only de minimis or biologically irrelevant amounts of other bacterial strains or species. Strikingly, the examples demonstrate that compositions comprising only a single strain of the invention can have potent effects (see e.g. Examples 1 and 3), with no reliance on other strains or species.

In some embodiments, the composition of the invention additionally comprises a bacterial strain that has the same safety and therapeutic efficacy characteristics as strain 42787, but which is not 42787, or which is not a Megasphaera massiliensis.

In some embodiments in which the composition of the invention comprises more than one bacterial strain, species or genus, the individual bacterial strains, species or genera may be for separate, simultaneous or sequential administration. For example, the composition may comprise all of the more than one bacterial strain, species or genera, or the bacterial strains, species or genera may be stored separately and be administered separately, simultaneously or sequentially. In some embodiments, the more than one bacterial strains, species or genera are stored separately but are mixed together prior to use.

In some embodiments, the bacterial strain for use in the invention is obtained from human adult faeces. In some embodiments in which the composition of the invention comprises more than one bacterial strain, all of the bacterial strains are obtained from human adult faeces or if other bacterial strains are present they are present only in de minimis amounts. The bacteria may have been cultured subsequent to being obtained from the human adult faeces and being used in a composition of the invention.

As mentioned above, in some embodiments, the one or more Megasphaera bacterial strains is/are the only therapeutically active agent(s) in a composition of the invention. In some embodiments, the bacterial strain(s) in the composition is/are the only therapeutically active agent(s) in a composition of the invention.

The compositions for use in accordance with the invention may or may not require marketing approval.

In certain embodiments, the invention provides the above pharmaceutical composition, wherein said bacterial strain is lyophilised. In certain embodiments, the invention provides the above pharmaceutical composition, wherein said bacterial strain is spray dried. In certain embodiments, the invention provides the above pharmaceutical composition, wherein the bacterial strain is lyophilised or spray dried and wherein it is live. In certain embodiments, the invention provides the above pharmaceutical composition, wherein the bacterial strain is lyophilised or spray dried and wherein it is viable. In certain embodiments, the invention provides the above pharmaceutical composition, wherein the bacterial strain is lyophilised or spray dried and wherein it is capable of partially or totally colonising the intestine. In certain embodiments, the invention provides the above pharmaceutical composition, wherein the bacterial strain is lyophilised or spray dried and wherein it is viable and capable of partially or totally colonising the intestine.

In some cases, the lyophilised bacterial strain is reconstituted prior to administration. In some cases, the reconstitution is by use of a diluent described herein.

The compositions of the invention can comprise pharmaceutically acceptable excipients, diluents or carriers.

In certain embodiments, the invention provides a pharmaceutical composition comprising: a bacterial strain of the invention; and a pharmaceutically acceptable excipient, carrier or diluent; wherein the bacterial strain is in an amount sufficient to treat or prevent a Gram-positive bacterial infection when administered to a subject in need thereof

In certain embodiments, the invention provides pharmaceutical composition comprising: a bacterial strain of the invention; and a pharmaceutically acceptable excipient, carrier or diluent; wherein the bacterial strain is in an amount sufficient to treat or prevent a Gram-positive bacterial infection.

In certain embodiments, the invention provides the above pharmaceutical composition, wherein the amount of the bacterial strain is from about 1×10³ to about 1×10¹¹ colony forming units per gram with respect to a weight of the composition.

In certain embodiments, the invention provides the above pharmaceutical composition, wherein the composition is administered at a dose of 1 g, 3 g, 5 g or 10 g.

In certain embodiments, the invention provides the above pharmaceutical composition, wherein the composition is administered by a method selected from the group consisting of oral, rectal, subcutaneous, nasal, buccal, and sublingual.

In certain embodiments, the invention provides the above pharmaceutical composition, comprising a carrier selected from the group consisting of lactose, starch, glucose, methyl cellulose, magnesium stearate, mannitol and sorbitol.

In certain embodiments, the invention provides the above pharmaceutical composition, comprising a diluent selected from the group consisting of ethanol, glycerol and water.

In certain embodiments, the invention provides the above pharmaceutical composition, comprising an excipient selected from the group consisting of starch, gelatin, glucose, anhydrous lactose, free-flow lactose, beta-lactose, corn sweetener, acacia, tragacanth, sodium alginate, carboxymethyl cellulose, polyethylene glycol, sodium oleate, sodium stearate, magnesium stearate, sodium benzoate, sodium acetate and sodium chloride.

In certain embodiments, the invention provides the above pharmaceutical composition, further comprising at least one of a preservative, an antioxidant and a stabilizer.

In certain embodiments, the invention provides the above pharmaceutical composition, comprising a preservative selected from the group consisting of sodium benzoate, sorbic acid and esters of p-hydroxybenzoic acid.

In certain embodiments, the invention provides the above pharmaceutical composition, wherein said bacterial strain is lyophilised.

In certain embodiments, the invention provides the above pharmaceutical composition, wherein when the composition is stored in a sealed container at about 4.0 or about 25.0 and the container is placed in an atmosphere having 50% relative humidity, at least 80% of the bacterial strain as measured in colony forming units, remains after a period of at least about: 1 month, 3 months, 6 months, 1 year, 1.5 years, 2 years, 2.5 years or 3 years.

In some embodiments, the composition of the invention is provided in a sealed container comprising a composition as described herein. In some embodiments, the sealed container is a sachet or bottle. In some embodiments, the composition of the invention is provided in a syringe comprising a composition as described herein.

The composition of the present invention may, in some embodiments, be provided as a pharmaceutical formulation. For example, the composition may be provided as a tablet or capsule. In some embodiments, the capsule is a gelatine capsule (“gel-cap”).

In some embodiments, the compositions of the invention are administered orally. Oral administration may involve swallowing, so that the compound enters the gastrointestinal tract, and/or buccal, lingual, or sublingual administration by which the compound enters the blood stream directly from the mouth.

Pharmaceutical formulations suitable for oral administration include solid plugs, solid microparticulates, semi-solid and liquid (including multiple phases or dispersed systems) such as tablets; soft or hard capsules containing multi- or nano-particulates, liquids (e.g. aqueous solutions), emulsions or powders; lozenges (including liquid-filled); chews; gels; fast dispersing dosage forms; films; ovules; sprays; and buccal/mucoadhesive patches.

In some embodiments the pharmaceutical formulation is an enteric formulation, i.e. a gastro-resistant formulation (for example, resistant to gastric pH) that is suitable for delivery of the composition of the invention to the intestine by oral administration. Enteric formulations may be particularly useful when the bacteria or another component of the composition is acid-sensitive, e.g. prone to degradation under gastric conditions.

In some embodiments, the enteric formulation comprises an enteric coating. In some embodiments, the formulation is an enteric-coated dosage form. For example, the formulation may be an enteric-coated tablet or an enteric-coated capsule, or the like. The enteric coating may be a conventional enteric coating, for example, a conventional coating for a tablet, capsule, or the like for oral delivery. The formulation may comprise a film coating, for example, a thin film layer of an enteric polymer, e.g. an acid-insoluble polymer.

In some embodiments, the enteric formulation is intrinsically enteric, for example, gastro-resistant without the need for an enteric coating. Thus, in some embodiments, the formulation is an enteric formulation that does not comprise an enteric coating. In some embodiments, the formulation is a capsule made from a thermogelling material. In some embodiments, the thermogelling material is a cellulosic material, such as methylcellulose, hydroxymethylcellulose or hydroxypropylmethylcellulose (HPMC). In some embodiments, the capsule comprises a shell that does not contain any film forming polymer. In some embodiments, the capsule comprises a shell and the shell comprises hydroxypropylmethylcellulose and does not comprise any film forming polymer (e.g. see [32]). In some embodiments, the formulation is an intrinsically enteric capsule (for example, Vcaps® from Capsugel).

In some embodiments, the formulation is a soft capsule. Soft capsules are capsules which may, owing to additions of softeners, such as, for example, glycerol, sorbitol, maltitol and polyethylene glycols, present in the capsule shell, have a certain elasticity and softness. Soft capsules can be produced, for example, on the basis of gelatine or starch. Gelatine-based soft capsules are commercially available from various suppliers. Depending on the method of administration, such as, for example, orally or rectally, soft capsules can have various shapes, they can be, for example, round, oval, oblong or torpedo-shaped. Soft capsules can be produced by conventional processes, such as, for example, by the Scherer process, the Accogel process or the droplet or blowing process.

Culturing Methods

The bacterial strains for use in the present invention can be cultured using standard microbiology techniques as detailed in, for example, references [33], [ ] and [35].

The solid or liquid medium used for culture may be YCFA agar or YCFA medium. YCFA medium may include (per 100 ml, approximate values): Casitone (1.0 g), yeast extract (0.25 g), NaHCO₃(0.4 g), cysteine (0.1 g), K₂HPO₄ (0.045 g), KH₂PO₄ (0.045 g), NaCl (0.09 g), (NH₄)₂SO₄ (0.09 g), MgSO₄.7H₂O (0.009 g), CaCl₂) (0.009 g), resazurin (0.1 mg), hemin (1 mg), biotin (1 μg), cobalamin (1 μg), p-aminobenzoic acid (3 μg), folic acid (5 μg), and pyridoxamine (15 μg).

Bacterial Strains for Use in Vaccine Compositions

The inventors have identified that the bacterial strains of the invention are useful for treating or preventing Gram-positive bacterial infections. This may be a result of the effect that the bacterial strains of the invention have on the host immune system. Therefore, the compositions of the invention may also be useful for preventing Gram-positive bacterial infections, when administered as vaccine compositions. In certain such embodiments, the bacterial strains of the invention may be killed, inactivated or attenuated. In certain such embodiments, the compositions may comprise a vaccine adjuvant. In certain embodiments, the compositions are for administration via injection, such as via subcutaneous injection.

General

The practice of the present invention will employ, unless otherwise indicated, conventional methods of chemistry, biochemistry, molecular biology, immunology and pharmacology, within the skill of the art. Such techniques are explained fully in the literature. See, e.g., references [36] and [37, 43], etc.

The term “comprising” encompasses “including” as well as “consisting” e.g. a composition “comprising” X may consist exclusively of X or may include something additional e.g. X+Y.

The term “about” in relation to a numerical value x is optional and means, for example, x+10%.

The word “substantially” does not exclude “completely” e.g. a composition which is “substantially free” from Y may be completely free from Y. Where necessary, the word “substantially” may be omitted from the definition of the invention.

References to a percentage sequence identity between two nucleotide sequences means that, when aligned, that percentage of nucleotides are the same in comparing the two sequences. This alignment and the percent homology or sequence identity can be determined using software programs known in the art, for example those described in section 7.7.18 of ref. [44]. A preferred alignment is determined by the Smith-Waterman homology search algorithm using an affine gap search with a gap open penalty of 12 and a gap extension penalty of 2, BLOSUM matrix of 62. The Smith-Waterman homology search algorithm is disclosed in ref [45].

Unless specifically stated, a process or method comprising numerous steps may comprise additional steps at the beginning or end of the method, or may comprise additional intervening steps. Also, steps may be combined, omitted or performed in an alternative order, if appropriate.

Various embodiments of the invention are described herein. It will be appreciated that the features specified in each embodiment may be combined with other specified features, to provide further embodiments. In particular, embodiments highlighted herein as being suitable, typical or preferred may be combined with each other (except when they are mutually exclusive).

### MODES FOR CARRYING OUT THE INVENTION

### Example 1—Strain 42787 Reduces Outgrowth of Gram-Positive B. subtilis

Introduction

The inventors sought to determine whether Strain 42787 could reduce the outgrowth of Gram-positive bacteria in an in vitro infection model.

Material and Methods

General

Agar plates and overnight culture broths were pre-equilibrated in an anaerobic cabinet for minimum 24 hours before use with anaerobic strains.

Agar plates and liquid media for use with aerobic strains was pre-warmed before use.

Infection Model

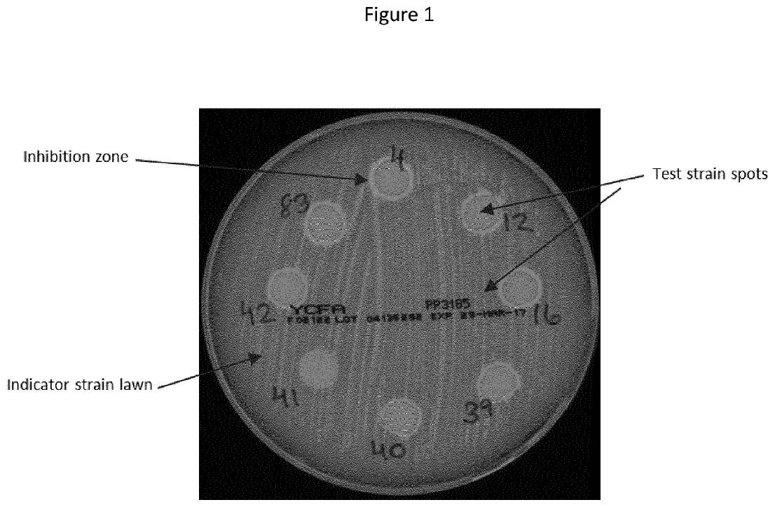

10 μl of strain 42787 culture was spot-inoculated (“test strain”, as shown on FIG. 1) on the upper surface of a pre-warmed YCFA agar plate. A negative control spot of 10 μl YCFA was also added. The inoculated YCFA plates were incubated in an anaerobic hood for 24-48 h to allow for growth of strain 42787.

Following, a test tube of 1% molten LBA (55° C.) was inoculated with 200 μl of an overnight culture of Gram-positive bacteria (Bacillus subtilis). The tube was vortexed for ten seconds. The inoculate was then poured over the surface of YCFA agar plate (the “indicator strain lawn”—FIG. 1). The plates were then incubated at 30° C. for a further 48 h under aerobic conditions.

Results

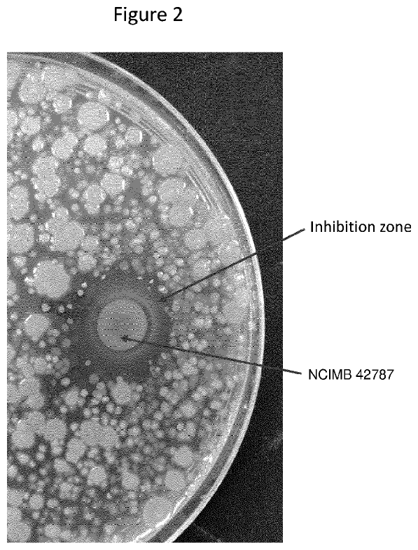

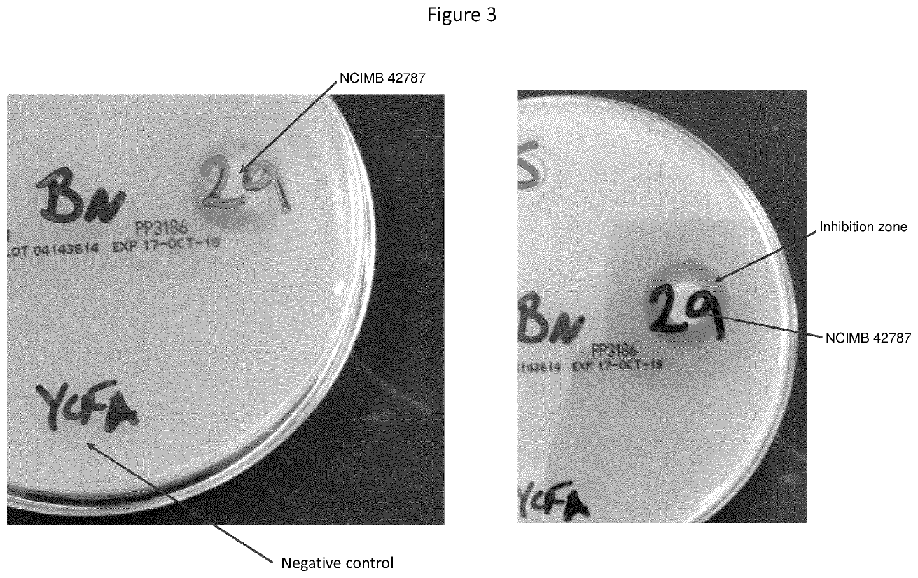

Antimicrobial activity against the B. subtilis was visualised by the presence of a growth inhibition zone around the strain 42787 spot (see FIG. 1). No growth inhibition was detected around the control well (See FIG. 3). Other test strains (FIG. 1—Strains 12, 41, 40, 39, etc.) exhibited minimal antimicrobial activity. The results show that strain 42787 inhibits the growth of Gram-positive bacteria (FIGS. 2 and 3).

### Example 2—M Massiliensis Strain NCIMB 42787 Produces Valeric Acid

Introduction

The gut microbiota, with its immense diversity and metabolic capacity, represents a huge metabolic reservoir for production of a vast variety of molecules. The inventors sought to determine what short chain fatty acids and medium chain fatty acids are produced by the M. massiliensis strain NCIMB 42787.

Material and Methods

Bacterial Culture and Cell-Free Supernatant Collection

Pure cultures of bacteria were grown anaerobically in YCFA broth until they reached their stationary growth phase. Cultures were centrifuged at 5,000×g for 5 minutes and the cell-free supernatant (CFS) was filtered using a 0.2 μM filter (Millipore, UK). 1 mL aliquots of the CFS were stored at −80° C. until use. Sodium butyrate, hexanoic and valeric acid were obtained from Sigma Aldrich (UK) and suspensions were prepared in YCFA broth.

SCFA and MCFA Quantification of Bacterial Supernatants

Short chain fatty acids (SCFAs) and medium chain fatty acids (MCFAs) from bacterial supernatants were analysed and quantified by MS Omics APS as follows. Samples were acidified using hydrochloride acid, and deuterium labelled internal standards where added. All samples were analysed in a randomized order. Analysis was performed using a high polarity column (Zebron™ ZB-FFAP, GC Cap. Column 30 m×0.25 mm×0.25 μm) installed in a GC (7890B, Agilent) coupled with a quadropole detector (59977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described in [46].

Results

Strain 42787 Produces Valeric Acid

Strain 42787 produced valeric acid and hexanoic acid at mean concentrations of 5.08 mM and 1.60 mM, respectively (FIG. 4). Valeric acid has been shown to reduce the viability of C. difficile [19]. The inventors also found other strains of the species M. massiliensis produces comparable levels of valeric acid, hexanoic acid, butyric acid and consume similar amounts of acetate and propionate (FIGS. 5 and 6).

### Example 3—Strain 42787 Reduces Outgrowth of Gram-Positive Clostridioides Difficile Hypervirulent RT 027

To investigate the potential inhibitory activity of valeric acid producing bacteria against a pathogenic Gram-positive bacteria, the inventors tested whether Strain 42787 could reduce the outgrowth of Clostridioides difficile hypervirulent RT 027 in an in vitro infection model

For the model, a lawn was created with a first generation broth culture revived from a Public Health England culture collection lyophilised stock (NCTC 13366). Once the pathogen was spread evenly onto brain heart infusion and YCFA agar, stationary-phase supernatants from Strain 42787 were spotted onto the plates in small volumes (10 μL).

The results showed that supernatant derived from Strain 42787 was effective in reducing outgrowth of Clostridioides difficile hypervirulent RT 027. This indicates that Strain 42787 has antimicrobial activity against pathogenic Gram-positive species.

### Example 4—Metabolite Analysis

Further to the data provided in Example 2, FIG. 7 demonstrates what other short chain fatty acids are produced and consumed by the M. massiliensis strain NCIMB 42787 and other strains deposited under accession numbers NCIMB 43385, NCIMB 43388 and NCIMB 43389.

M. massiliensis strain NCIMB 42787 reduces formic acid while increasing levels of 2-methyl-propanoic and 3-methyl-propanoic acid (FIG. 7). Therefore, strain NCIMB 42787 produces 2-methyl-propanoic and 3-methyl-propanoic acid and consumes formic acid. The inventors also found that other of the deposited strains produce comparable levels of 2-methyl-propanoic and 3-methyl-propanoic acid and consume similar amounts of formic acid.

### Example 5—Effect of Megasphaera Massiliensis Strain DSM 26228, Megasphaera elsdenii Strain NCIMB 8927 and Megasphaera Massiliensis Strain NCIMB 42787 on Short-Chain Fatty Acid Production In Vitro

Summary

This study investigated the effect of DSM 26228, NCIMB 8927 and NCIMB 42787 on the production of short-chain fatty acids (SCFAs) in vitro. SCFAs, which include acetate, propionate, valerate, isobutyrate and isovalerate are microbial by-products of dietary fibre. An increase in any SCFA suggests an increase in productivity of the microbiota and is a desirable trait.

Material and Methods

Pure cultures of DSM 26228, NCIMB 8927 and NCIMB 42787 were grown anaerobically in YCFA+broth [Per litre: Casein hydrolysate 10.0 g, Yeast Extract 2.5 g, Sodium hydrogen carbonate 4.0 g, Glucose 2.0 g, Cellobiose 2.0 g, Soluble starch 2.0 g, Di-potassium hydrogen phosphate 0.45 g, Potassium di-hydrogen phosphate 0.45 g, Resazurin 0.001 g, L-Cysteine HCl 1.0 g, Ammonium sulphate 0.9 g, Sodium chloride 0.9 g, Magnesium sulphate 0.09 g, Calcium chloride 0.09 g, Haemin 0.01 g, SCFA 3.1 ml (Acetic acid 2.026 ml/L, Propionic acid 0.715 ml/L, n-Valeric acid 0.119 ml/L, Iso-Valeric acid 0.119 ml/L, Iso-Butyric acid 0.119 ml/L), vitamin mix 1:1 ml (Biotin 1 mg/100 ml, Cyanocobalamine 1 mg/100 ml, p-Aminobenzoic acid 3 mg/100 ml, Pyridoxine 15 mg/100 ml), vitamin mix 2:1 ml (Thiamine 5 mg/100 ml, Riboflavin 5 mg/100 ml), vitamin mix 3:1 ml (Folic acid 5 mg/100 ml)] until they reached their stationary growth phase. Cultures were centrifuged at 5000×g for 10 minutes and the cell-free supernatant (CFS) was filtered using a 0.45 μM followed by a 0.2 μM filter (Millipore, UK), after which 1 mL aliquots of the CFS were stored at −80° C. until use.

Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25 μm) installed in a gas chromatograph (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software described by reference [46].

Results

The following pattern of was observed for each of the bacterial strains:

| | | | | 2-methyl- | | 3-methyl- | | 4-methyl- | | |
|---|---|---|---|---|---|---|---|---|---|---|
| | Acetic | Formic | Propanoic | propanoic | Butanoic | butanoic | Pentanoic | pentanoic | Hexanoic | Heptanoic |
| | acid | acid | acid | acid | acid | acid | acid | acid | acid | acid |
| DSM | −17.6 | −0.4 | −4.9 | 1.7 | 16.0 | 1.5 | 5.8 | 0.0 | 1.6 | 0.1 |
| 26228 | | | | | | | | | | |
| NCIMB | −2.4 | −0.1 | −2.5 | 0.2 | 10.7 | 0.5 | 2.8 | 0.0 | 0.3 | 0.1 |
| 8927 | | | | | | | | | | |
| NCIMB | −20.2 | −0.4 | −5.6 | 2.1 | 15.8 | 4.5 | 6.6 | 0.0 | 2.2 | 0.1 |
| 42787 | | | | | | | | | | |

These data demonstrate that all three strains of Megasphaera increase butyrate (butanoic acid) and valeric acid (pentanoic acid).

As outlined above, valeric acid reduces the viability of pathogenic Gram-positive bacteria. Therefore, bacterial strains which increase valeric acid are useful in treating or preventing Gram-positive bacterial infections. Both Megasphaera massiliensis and Megasphaera elsdenii strains trigger beneficial increases in valeric acid. Therefore, in certain embodiments, the compositions of the present invention demonstrate efficacy in treating or preventing Gram-positive bacterial infections, in particular pathogenic Gram-positive bacterial infections, in light of the increase in valeric acid.

Butyrate attenuates intestinal inflammation and improves intestinal barrier function, for example by increasing expression of components of epithelial cell tight junctions (via activation of the HIF-1 transcription factor). Accordingly, butyrate protects intestinal epithelial cells from damage caused by Gram-positive bacterial toxins by stabilising the intestinal barrier, mitigating local inflammatory responses and systemic consequences of Gram-positive pacterial infection. Accordingly, bacterial strains that increase levels of butyrate are useful in the treatment of and/or prevention of symptoms associated with Gram-positive bacterial infection, for example C. difficile infection. Both Megasphaera massiliensis and Megasphaera elsdenii strains trigger beneficial increases in butyrate. In certain embodiments, the compositions of the present invention demonstrate therapeutic efficacy in treating or preventing Gram-positive bacterial infection, or treating or preventing the symptoms associated with Gram-positive bacterial infection, in light of the increase in butyrate.

### Example 6—Megasphaera Massiliensis Strain Deposited Under Accession Number NCIMB 42787 Significantly Reduces the Efficacy of TNFα Production in Splenocytes of BALB/c Mice in Response to Antigenic Challenge

Materials and Methods

BALBc (Envigo, UK) adult male mice were group housed under a 12 h light-dark cycle; standard rodent chow and water were available ad libitum. All experiments were performed in accordance with European guidelines. Animals were 8 weeks old at the start of the experiment.

Animals were allowed to habituate to their holding room for one week after arrival into the animal unit. The mice received oral gavage (200 μL dose) of NCIMB 42787 as a live biotherapeutic at a dose of 1×10⁹ CFU for 6 consecutive days between 15:00 and 17:00. On day 7, the animals are decapitated and tissues are harvested for experimentation. The spleen was removed, collected in 5 mL RPMI media (with L-glutamine and sodium bicarbonate, R8758 Sigma+10% FBS (F7524, Sigma)+1% Pen/Strep (P4333, Sigma)) and processed immediately after culls for ex-vivo immune stimulation.

A spleen cytokine assay was used to quantify the peripheral levels of pro-inflammatory markers, for example TNFα. Spleens were collected immediately in 5 mL RPMI media following sacrifice and cultured immediately. Spleen cells were first homogenised in the RPMI media. The homogenate step was followed by RBC lysis step where the cells were incubated for 5 mins in 1 ml of RBC lysis buffer (11814389001 ROCHE, Sigma). 10 ml of the media was added to stop the lysis and followed by 200 g centrifugation for 5 mins. This was followed by final step where the cells were passed through 40 μm strainer. The homogenate was then filtered over a 40 μm strainer, centrifuged at 200 g for 5 min and resuspended in media. Cells were counted and seeded (4,000,000/mL media). After 2.5 h of adaptation, cells were stimulated with concanavalin A (ConA-2.5 μg/ml) for 24 h. Following stimulation, the supernatants were harvested to assess the cytokine release using Proinflammatory Panel 1 (mouse) V-PLEX Kit (Meso Scale Discovery, Maryland, USA) for TNFα. The analyses were performed using MESO QuickPlex SQ 120, SECTOR Imager 2400, SECTOR Imager 6000, SECTOR S 600.

Results

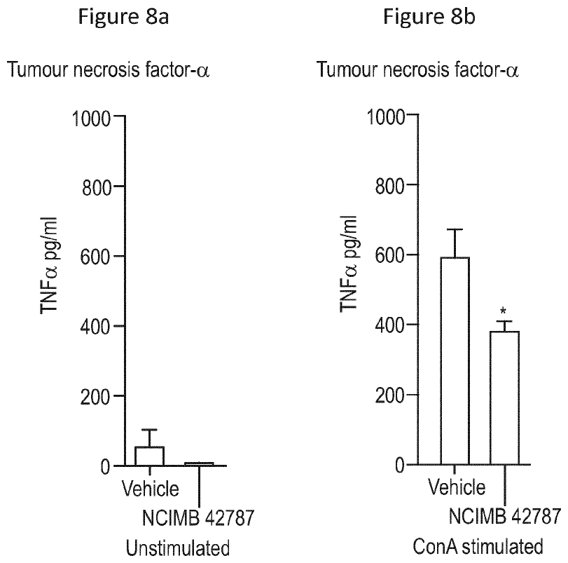

FIG. 8B demonstrates that splenocytes isolated from mice administered NCIMB 42787 produced significantly reduced levels of TNFα after ConA stimulation compared to those mice treated with a vehicle control. In addition, administration of NCIMB 42787 alone does change the efficacy of TNFα production by splenocytes in the absence of stimulation (FIG. 8A).

Conclusion

Megasphaera massiliensis strain NCIMB 42787 dampens the pro-inflammatory response triggered by splenocytes, in particular by reducing the efficacy of TNFα production by these cells. Both local and systemic inflammatory processes are involved in C. difficile infection, with the latter manifesting clinically as fever. TNFα is implicated as a key player in the systemic inflammatory response in C. difficile infection, and is associated with poor prognosis in patients with C. difficile infection. Therefore, administration of Megasphaera massiliensis would reduce TNFα production and dampen the systemic inflammatory response, providing a therapeutic benefit for patients with C. difficile infection.

Accordingly, not only do the strains of the invention or for use in the invention have bacteriocidal properties to treat or prevent C. difficile infection, they are able to improve the symptoms of patients with the infection. Therefore, in some embodiments, the composition of the invention is for use in the treatment or prevention of C. difficile infection (CDI). In some embodiments, the composition of the invention is for use in the treatment of and/or prevention of symptoms associated with CDI. In some embodiments, the composition of the invention treats or prevents symptoms associated with CDI by reducing the inflammatory response, in particular by reducing the production of TNFα.

### Example 7—Megasphaera Massiliensis Strain NCIMB 43389 and Megasphaera Sp. Strain NCIMB 43385 Increase E-Cadherin and Occludin Levels in Colon Epithelial Cell Lines

Materials and Methods

Cells (HT29 and HCT116) were seeded in black 96 well plates at a density of 10,000 cells/well overnight and were treated with 10% bacterial supernatant for 24 h. Afterwards, the cells were fixed with 4% paraformaldehyde in PBS (pH 7.3) for 20 min at room temperature (RT). Fixed cells were washed with PBS, and permeabilized with 0.5% Triton X-100 in PBS for 10 min After washing with PBS, the plates were incubated with blocking buffer (4% BSA/PBS) for 1 h at RT before adding the primary antibody (anti-Occludin (71-1500; ThermoFisher), 1:200 diluted in 1% BSA/PBS for 12 h at 4° C., or anti-E-cadherin (13-1700, ThermoFisher), 1:1000 diluted in 1% BSA/PBS for 1 hat 4° C.). They were then washed twice with PBS, followed by incubation with Alexa Flour 488 conjugated anti-rabbit (Molecular Probes Inc) and Alexa Flour 594 ((Molecular Probes Inc) conjugated for 1 h at RT. After washing 3× with PBS, the plates were labelled with DAPI and washed with PBS 3×. Plates were viewed using ImageExpress PIco microscope (Molecular Devices) equipped with a 20× objective and filter sets suitable for detection of the fluorochromes used. Stored images were saved as TIFF files. Raw analysis data generated by the PICO analysis module were plotted and analysed using GraphPad Prism 7 software. Representative images were selected to illustrate the differences in abundance and location of the proteins examined

Results

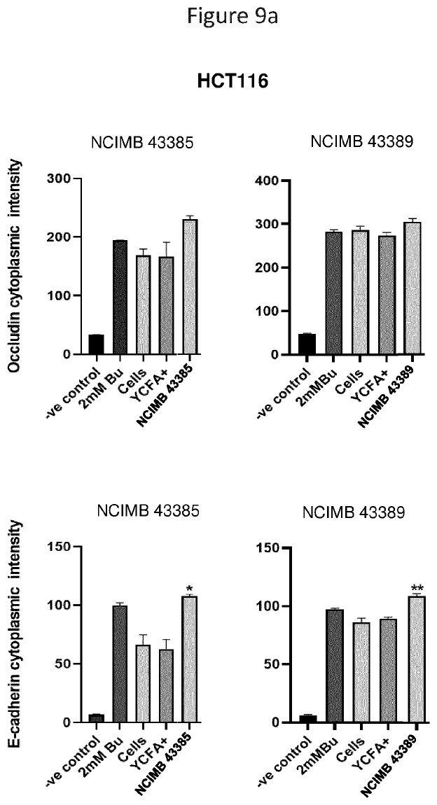

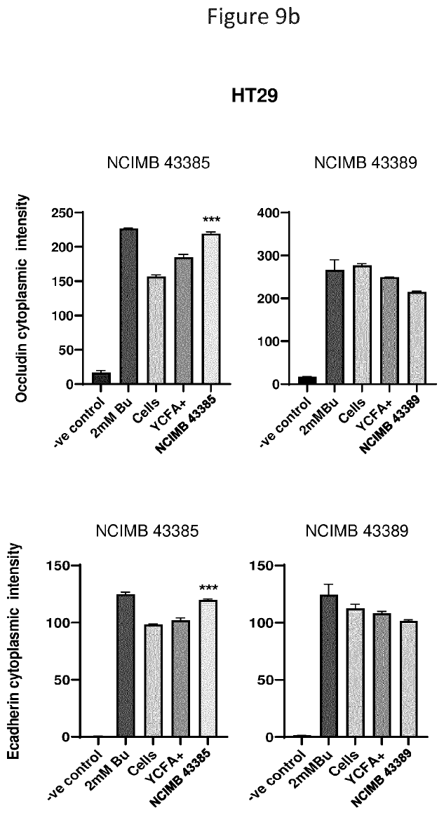

FIG. 9a shows that for HCT116 cells, cells treated with supernatants of Megasphaera sp. strain NCIMB 43385 or Megasphaera massiliensis strain NCIMB 43389 have higher levels of occludin and E-cadherin compared to both untreated cells and cells treated with YCFA+medium alone. FIG. 9b shows that for HT29 cells, cells treated with supernatants of Megasphaera sp. strain NCIMB 43385 have higher levels of occludin and E-cadherin compared to both untreated cells and cells treated with YCFA+medium alone.

Conclusion

E-cadherin and occludin are both transmembrane proteins that are involved in cell-cell adhesion, with occludin specifically being a component of epithelial tight junctions [47, 48]. The results show that supernatants of Megasphaera strains are able to upregulate these proteins in HCT116 and HT29 cells, which are both human colon epithelial cell lines. Taken together with Example 5, these results support the conclusion that Megasphaera strains can improve intestinal barrier function, e.g. through butyrate signalling. Accordingly, this provides further evidence that Megasphaera strains may be useful in protecting intestinal epithelial cells from damage caused by Gram-positive bacterial toxins.

### Example 8—Short/Medium Chain Fatty Acid Production Profile of Megasphaera Massiliensis Strain NCIMB 43389 and Megasphaera Sp. Strain NCIMB 43385

Materials and Methods

Pure cultures of Megasphaera massiliensis strain NCIMB 43389 and Megasphaera sp. strain NCIMB 43385 were grown anaerobically in YCFA+broth. Short chain fatty acids (SCFAs) and medium chain fatty acids (MCFAs) from bacterial supernatants were analysed and quantified by MS Omics APS, Denmark. Samples were acidified using hydrochloride acid, and deuterium labelled internal standards were added. All samples were analyzed in a randomized order. Analysis was performed using a high polarity column (Zebron™ ZB-FFAP, GC Cap. Column 30 m×0.25 mm×0.25 μm) installed in a gas chromatograph (7890B, Agilent) coupled with a quadropole detector (5977B, Agilent). The system was controlled by ChemStation (Agilent). Raw data was converted to netCDF format using Chemstation (Agilent), before the data was imported and processed in Matlab R2014b (Mathworks, Inc.) using the PARADISe software.

Results

| | Change in short/medium chain fatty acid concentration (mM) | | | | | | |
|---|---|---|---|---|---|---|---|
| | Succinic | Formic | Acetic | Propionic | Butyric | Valeric | Hexanoic |
| Strain | acid | acid | acid | acid | acid | acid | acid |
| Megasphaera | Not | 1.51 | −16 | −5.25 | 27.86 | 6.44 | 0.96 |
| massiliensis | detected | | | | | | |
| NCIMB 43389 | | | | | | | |
| Megasphaera sp. | Not | Not | 7.73 | −1.07 | 16.28 | 5.94 | 1.22 |
| strain NCIMB 43385 | detected | detected | | | | | |

The results are consistent with those reported in Example 5 (and FIGS. 5-7) for Megasphaera massiliensis strain NCIMB 43389 and Megasphaera sp. strain NCIMB 43385, further confirming the short/medium fatty acid chain profile of these strains.

| Sequences | |
|---|---|
| (Megasphaera massihensis gene for 16S ribosomal RNA, partial sequence, | |
| strain: NP3 - JX424772.1) | |
| SEQ ID NO: 1 | |
| 1 | agagtttgat cctggctcag gacgaacgct ggcggcgtgc ttaacacatg caagtcgaac |
| 61 | gagaagagat gagaagcttg cttcttatca attcgagtgg caaacgggtg agtaacgcgt |
| 121 | aagcaacctg cccttcagat ggggacaaca gctggaaacg gctgctaata ccgaatacgt |
| 181 | tctttccgcc gcatgacggg aagaagaaag ggaggccttc gggctttcgc tggaggaggg |
| 241 | gcttgcgtct gattagctag ttggaggggt aacggcccac caaggcgacg atcagtagcc |
| 301 | ggtctgagag gatgaacggc cacattggga ctgagacacg gcccagactc ctacgggagg |
| 361 | cagcagtggg gaatcttccg caatggacga aagtctgacg gagcaacgcc gcgtgaacga |
| 421 | tgacggcctt cgggttgtaa agttctgtta tatgggacga acagggcatc ggttaatacc |
| 481 | cggtgtcttt gacggtaccg taagagaaag ccacggctaa ctacgtgcca gcagccgcgg |
| 541 | taatacgtag gtggcaagcg ttgtccggaa ttattgggcg taaagggcgc gcaggcggca |
| 601 | tcgcaagtcg gtcttaaaag tgcggggctt aaccccgtga ggggaccgaa actgtgaagc |
| 661 | tcgagtgtcg gagaggaaag cggaattcct agtgtagcgg tgaaatgcgt agatattagg |
| 721 | aggaacacca gtggcgaaag cggctttctg gacgacaact gacgctgagg cgcgaaagcc |
| 781 | aggggagcaa acgggattag ataccccggt agtcctggcc gtaaacgatg gatactaggt |
| 841 | gtaggaggta tcgactcctt ctgtgccgga gttaacgcaa taagtatccc gcctggggag |
| 901 | tacggccgca aggctgaaac tcaaaggaat tgacgggggc ccgcacaagc ggtggagtat |
| 961 | gtggtttaat tcgacgcaac gcgaagaacc ttaccaagcc ttgacattga ttgctacgga |
| 1021 | aagagatttc cggttcttct tcggaagaca agaaaacagg tggtgcacgg ctgtcgtcag |
| 1081 | ctcgtgtcgt gagatgttgg gttaagtccc gcaacgagcg caacccctat cttctgttgc |
| 1141 | cagcacctcg ggtggggact cagaagagac tgccgcagac aatgcggagg aaggcgggga |
| 1201 | tgacgtcaag tcatcatgcc ccttatggct tgggctacac acgtactaca atggctctta |
| 1261 | atagagggac gcgaaggagc gatccggagc aaaccccaaa aacagagtcc cagttcggat |
| 1321 | tgcaggctgc aactcgcctg catgaagcag gaatcgctag taatcgcagg tcagcatact |
| 1381 | gcggtgaata cgttcccggg ccttgtacac accgcccgtc acaccacgaa agtcattcac |
| 1441 | acccgaagcc ggtgaggcaa ccgcaaggaa ccagccgtcg aaggtggggg cgatgattgg |
| 1501 | ggtgaagtcg taacaaggt |

(consensus 16S rRNA sequence for Megasphaera massihensis strain 42787)
SEQ ID NO: 2
TGAGAAGCTTGCTTCTTATCGATTCTAGTGGCAAACGGGTGAGTAACGCGTAAGCAACC
TGCCCTTCAGATGGGGACAACAGCTGGAAACGGCTGCTAATACCGAATACGTTCTTTCC
GCCGCATGACGGGAAGAAGAAAGGGAGGCCTTCGGGCTTTCGCTGGAGGAGGGGCTTG
CGTCTGATTAGCTAGTTGGAGGGGTAACGGCCCACCAAGGCGACGATCAGTAGCCGGTC
TGAGAGGATGAACGGCCACATTGGGACTGAGACACGGCCCAGACTCCTACGGGAGGCA
GCAGTGGGGAATCTTCCGCAATGGACGAAAGTCTGACGGAGCAACGCCGCGTGAACGA
TGACGGCCTTCGGGTTGTAAAGTTCTGTTATATGGGACGAACAGGACATCGGTTAATAC
CCGGTGTCTTTGACGGTACCGTAAGAGAAAGCCACGGCTAACTACGTGCCAGCAGCCGC
GGTAATACGTAGGTGGCAAGCGTTGTCCGGAATTATTGGGCGTAAAGGGCGCGCAGGC
GGCATCGCAAGTCGGTCTTAAAAGTGCGGGGCTTAACCCCGTGAGGGGACCGAAACTGT
GAAGCTCGAGTGTCGGAGAGGAAAGCGGAATTCCTAGTGTAGCGGTGAAATGCGTAGA
TATTAGGAGGAACACCAGTGGCGAAAGCGGCTTTCTGGACGACAACTGACGCTGAGGC
GCGAAAGCCAGGGGAGCAAACGGGATTAGATACCCCGGTAGTCCTGGCCGTAAACGAT
GGATACTAGGTGTAGGAGGTATCGACTCCTTCTGTGCCGGAGTTAACGCAATAAGTATC
CCGCCTGGGGAGTACGGCCGCAAGGCTGAAACTCAAAGGAATTGACGGGGGCCCGCAC
AAGCGGTGGAGTATGTGGTTTAATTCGACGCAACGCGAAGAACCTTACCAAGCCTTGAC
ATTGATTGCTACGGAAAGAGATTTCCGGTTCTTCTTCGGAAGACAAGAAAACAGGTGGT
GCACGGCTGTCGTCAGCTCGTGTCGTGAGATGTTGGGTTAAGTCCCGCAACGAGCGCAA
CCCCTATCTTCTGTTGCCAGCACCTCGGGTGGGGACTCAGAAGAGACTGCCGCAGACAA
TGCGGAGGAAGGCGGGGATGACGTCAAGTCATCATGCCCCTTATGGCTTGGGCTACACA
CGTACTACAATGGCTCTTAATAGAGGGAAGCGAAGGAGCGATCCGGAGCAAACCCCAA
AAACAGAGTCCCAGTTCGGATTGCAGGCTGCAACTCGCCTGCATGAAGCAGGAATCGCT
AGTAATCGCAGGTCAGCATACTGCGGTGAATACGTTCCCGGGCCTTGTACACACCGCCC
GTCACACCACGAAAGTCATTCACACCCGAAGCCGGTGAGGCAACCGCAAG
(consensus 16S rRNA sequence for the Megasphaera strain deposited
under accession number NCIMB 43385)
SEQ ID NO: 3
GGCTGGTTCCTTGCGGTTGCCTCACCGGCTTCGGGTGTGAATGACTTTCGTGGTGTGACG
GGCGGTGTGTACAAGGCCCGGGAACGTATTCACCGCAGTATGCTGACCTGCGATTACTA
GCGATTCCTGCTTCATGCAGGCGAGTTGCAGCCTGCAATCCGAACTGGGACTCTGTTTTT
GGGGTTTGCTCCGGATCGCTCCTTCGCTTCCCTCTATTAAGAGCCATTGTAGTACGTGTG
TAGCCCAAGCCATAAGGGGCATGATGACTTGACGTCATCCCCGCCTTCCTCCGCATTGTC
TGCGGCAGTCTCTTCTGAGTCCCCACCCTTAGTGCTGGCAACAGAAGATAGGGGTTGCG
CTCGTTGCGGGACTTAACCCAACATCTCACGACACGAGCTGACGACAGCCGTGCACCAC
CTGTTTTCTTGTCTTCCGAAGAAGAACCGGAAATCTCTTTCCGTAGCAATCAATGTCAAG
GCTTGGTAAGGTTCTTCGCGTTGCGTCGAATTAAACCACATACTCCACCGCTTGTGCGGG
CCCCCGTCAATTCCTTTGAGTTTCAGCCTTGCGGCCGTACTCCCCAGGCGGGATACTTAT
TGCGTTAACTCCGGCACAGAAGGAGTCGATACCTCCTACACCTAGTATCCATCGTTTACG
GCCAGGACTACCGGGGTATCTAATCCCGTTTGCTCCCCTGGCTTTCGCGCCTCAGCGTCA
GTTGTCGTCCAGAAAGCCGCTTTCGCCACTGGTGTTCCTCCTAATATCTACGCATTTCAC
CGCTACACTAGGAATTCCGCTTTCCTCTCCGACACTCGAGCTTCACAGTTTCGGTCCCCT
CACGGGGTTAAGCCCCGCACTTTTAAGACCGACTTGCGATGCCGCCTGCGCGCCCTTTAC
GCCCAATAATTCCGGACAACGCTTGCCACCTACGTATTACCGCGGCTGCTGGCACGTAG
TTAGCCGTGGCTTTCTCTTACGGTACCGTCAGGGATAACGGGTATTGACCGCTATCCTGT
TCGTCCCATATAACAGAACTTTACAACCCGAAGGCCGTCATCGTTCACGCGGCGTTGCTC
CGTCAGACTTTCGTCCATTGCGGAAGATTCCCCACTGCTGCCTCCCGTAGGAGTCTGGGC
CGTGTCTCAGTCCCAATGTGGCCGTTCATCCTCTCAGACCGGCTACTGATCGTCGCCTTG
GTGGGCCGTTACCCCTCCAACTAGCTAATCAGACGCAAGCCCCTCCTCCAGCGAAAGCC
CGAAGGCCTCCCTTTCTTCATCCCGTCATGCGGCGGAAAGAACGTATTCGGTATTAGCA
GCCGTTTCCAGCTGTTGTCCCCATCTGAAGGGCAGGTTGCTTACGCGTTACTCACCCGTT
TGCCACTCGAATTGATAAGAAGCAAGCTTCTCATC
(consensus 16S rRNA sequence for the Megasphaera massilliensis strain
deposited under accession number NCIMB 43388)
SEQ ID NO: 4
GGCTGGTTCCTTGCGGTTGCCTCACCGGCTTCGGGTGTGAATGACTTTCGTGGTGTGACG
GGCGGTGTGTACAAGGCCCGGGAACGTATTCACCGCAGTATGCTGACCTGCGATTACTA
GCGATTCCTGCTTCATGCAGGCGAGTTGCAGCCTGCAATCCGAACTGGGACTCTGTTTTT
GGGGTTTGCTCCGGATCGCTCCTTCGCTTCCCTCTATTAAGAGCCATTGTAGTACGTGTG
TAGCCCAAGCCATAAGGGGCATGATGACTTGACGTCATCCCCGCCTTCCTCCGCATTGTC
TGCGGCAGTCTCTTCTGAGTCCCCACCCGAGGTGCTGGCAACAGAAGATAGGGGTTGCG
CTCGTTGCGGGACTTAACCCAACATCTCACGACACGAGCTGACGACAGCCGTGCACCAC
CTGTTTTCTTGTCTTCCGAAGAAGAACCGGAAATCTCTTTCCGTAGCAATCAATGTCAAG
GCTTGGTAAGGTTCTTCGCGTTGCGTCGAATTAAACCACATACTCCACCGCTTGTGCGGG
CCCCCGTCAATTCCTTTGAGTTTCAGCCTTGCGGCCGTACTCCCCAGGCGGGATACTTAT
TGCGTTAACTCCGGCACAGAAGGAGTCGATACCTCCTACACCTAGTATCCATCGTTTACG
GCCAGGACTACCGGGGTATCTAATCCCGTTTGCTCCCCTGGCTTTCGCGCCTCAGCGTCA
GTTGTCGTCCAGAAAGCCGCTTTCGCCACTGGTGTTCCTCCTAATATCTACGCATTTCAC
CGCTACACTAGGAATTCCGCTTTCCTCTCCGACACTCGAGCTTCACAGTTTCGGTCCCCT
CACGGGGTTAAGCCCCGCACTTTTAAGACCGACTTGCGATGCCGCCTGCGCGCCCTTTAC
GCCCAATAATTCCGGACAACGCTTGCCACCTACGTATTACCGCGGCTGCTGGCACGTAG
TTAGCCGTGGCTTTCTCTTACGGTACCGTCAAAGACACCGGGTATTAACCGATGTCCTGT
TCGTCCCATATAACAGAACTTTACAACCCGAAGGCCGTCATCGTTCACGCGGCGTTGCTC
CGTCAGACTTTCGTCCATTGCGGAAGATTCCCCACTGCTGCCTCCCGTAGGAGTCTGGGC
CGTGTCTCAGTCCCAATGTGGCCGTTCATCCTCTCAGACCGGCTACTGATCGTCGCCTTG
GTGGGCCGTTACCCCTCCAACTAGCTAATCAGACGCAAGCCCCTCCTCCAGCGAAAGCC
CGAAGGCCTCCCTTTCTTCTTCCCGTCATGCGGCGGAAAGAACGTATTCGGTATTAGCAG
CCGTTTCCAGCTGTTGTCCCCATCTGAAGGGCAGGTTGCTTACGCGTTACTCACCCGTTT
GCCACTAGAATCGATAAGAAGCAAGCTTCTCATGTCTTCT
(consensus 16S rRNA sequence for the Megasphaera massilliensis strain
deposited under accession number NCIMB 43389)
SEQ ID NO: 5
CGACGGCTGGTTCCTTGCGGTTGCCTCACCGGCTTCGGGTGTGAATGACTTTCGTGGTGT
GACGGGCGGTGTGTACAAGGCCCGGGAACGTATTCACCGCAGTATGCTGACCTGCGATT
ACTAGCGATTCCTGCTTCATGCAGGCGAGTTGCAGCCTGCAATCCGAACTGGGACTCTG
TTTTTGGGGTTTGCTCCGGATCGCTCCTTCGCTTCCCTCTATTAAGAGCCATTGTAGTACG
TGTGTAGCCCAAGCCATAAGGGGCATGATGACTTGACGTCATCCCCGCCTTCCTCCGCAT
TGTCTGCGGCAGTCTCTTCTGAGTCCCCACCCGAGGTGCTGGCAACAGAAGATAGGGGT
TGCGCTCGTTGCGGGACTTAACCCAACATCTCACGACACGAGCTGACGACAGCCGTGCA
CCACCTGTTTTCTTGTCTTCCGAAGAAGAACCGGAAATCTCTTTCCGTAGCAATCAATGT
CAAGGCTTGGTAAGGTTCTTCGCGTTGCGTCGAATTAAACCACATACTCCACCGCTTGTG
CGGGCCCCCGTCAATTCCTTTGAGTTTCAGCCTTGCGGCCGTACTCCCCAGGCGGGATAC
TTATTGCGTTAACTCCGGCACAGAAGGAGTCGATACCTCCTACACCTAGTATCCATCGTT
TACGGCCAGGACTACCGGGGTATCTAATCCCGTTTGCTCCCCTGGCTTTCGCGCCTCAGC
GTCAGTTGTCGTCCAGAAAGCCGCTTTCGCCACTGGTGTTCCTCCTAATATCTACGCATT
TCACCGCTACACTAGGAATTCCGCTTTCCTCTCCGACACTCGAGCTTCACAGTTTCGGTC
CCCTCACGGGGTTAAGCCCCGCACTTTTAAGACCGACTTGCGATGCCGCCTGCGCGCCCT
TTACGCCCAATAATTCCGGACAACGCTTGCCACCTACGTATTACCGCGGCTGCTGGCAC
GTAGTTAGCCGTGGCTTTCTCTTACGGTACCGTCAAAGACACCGGGTATTAACCGATGCC
CTGTTCGTCCCATATAACAGAACTTTACAACCCGAAGGCCGTCATCGTTCACGCGGCGTT
GCTCCGTCAGACTTTCGTCCATTGCGGAAGATTCCCCACTGCTGCCTCCCGTAGGAGTCT
GGGCCGTGTCTCAGTCCCAATGTGGCCGTTCATCCTCTCAGACCGGCTACTGATCGTCGC
CTTGGTGGGCCGTTACCCCTCCAACCAGCTAATCAGACGCAAGCCCCTCCTCCAGCGAA
AGCCCGAAGGCCTCCCTTTCTTCTTCCCGTCATGCGGCGGAAAGAACGTATTCGGTATTA
GCAGCCGTTTCCAGCTGTTGTCCCCATCTGAAGGGCAGGTTGCTTACGCGTTACTCACCC
GTTTGCCACTAGAATCGATAAGAAGCAAGCTTCTCATGTCTTCTCGTTCGACTTGCAT
(consensus 16S rRNA sequence for the Megasphaera strain deposited
under accession number NCIMB 43386)
SEQ ID NO: 6
CGACGGCTGGTTCCTTGCGGTTGCCTCACCGGCTTCGGGTGTGAATGACTTTCGTGGTGT
GACGGGCGGTGTGTACAAGGCCCGGGAACGTATTCACCGCAGTATGCTGACCTGCGATT
ACTAGCGATTCCTGCTTCATGCAGGCGAGTTGCAGCCTGCAATCCGAACTGGGACTCTG
TTTTTGGGGTTTGCTCCGGATCGCTCCTTCGCTTCCCTCTATTAAGAGCCATTGTAGTACG
TGTGTAGCCCAAGCCATAAGGGGCATGATGACTTGACGTCATCCCCGCCTTCCTCCGCAT
TGTCTGCGGCAGTCTCTTCTGAGTCCCCACCCTTAGTGCTGGCAACAGAAGATAGGGGTT
GCGCTCGTTGCGGGACTTAACCCAACATCTCACGACACGAGCTGACGACAGCCGTGCAC
CACCTGTTTTCTTGTCTTCCGAAGAAGAACCGGAAATCTCTTTCCGTAGCAATCAATGTC
AAGGCTTGGTAAGGTTCTTCGCGTTGCGTCGAATTAAACCACATACTCCACCGCTTGTGC
GGGCCCCCGTCAATTCCTTTGAGTTTCAGCCTTGCGGCCGTACTCCCCAGGCGGGATACT
TATTGCGTTAACTCCGGCACAGAAGGAGTCGATACCTCCTACACCTAGTATCCATCGTTT
ACGGCCAGGACTACCGGGGTATCTAATCCCGTTTGCTCCCCTGGCTTTCGCGCCTCAGCG
TCAGTTGTCGTCCAGAAAGCCGCTTTCGCCACTGGTGTTCCTCCTAATATCTACGCATTT
CACCGCTACACTAGGAATTCCGCTTTCCTCTCCGACACTCGAGCTTCACAGTTTCGGTCC
CCTCACGGGGTTAAGCCCCGCACTTTTAAGACCGACTTGCGATGCCGCCTGCGCGCCCTT
TACGCCCAATAATTCCGGACAACGCTTGCCACCTACGTATTACCGCGGCTGCTGGCACG
TAGTTAGCCGTGGCTTTCTCTTACGGTACCGTCAGGGATAACGGGTATTGACCGCTATCC
TGTTCGTCCCATATAACAGAACTTTACAACCCGAAGGCCGTCATCGTTCACGCGGCGTTG
CTCCGTCAGACTTTCGTCCATTGCGGAAGATTCCCCACTGCTGCCTCCCGTAGGAGTCTG
GGCCGTGTCTCAGTCCCAATGTGGCCGTTCATCCTCTCAGACCGGCTACTGATCGTCGCC
TTGGTGGGCCGTTACCCCTCCAACTAGCTAATCAGACGCAAGCCCCTCCTCCAGCGAAA
GCCCGAAGGCCTCCCTTTCTTCATCCCGTCATGCGGCGGAAAGAACGTATTCGGTATTAG
CAGCCGTTTCCAGCTGTTGTCCCCATCTGAAGGGCAGGTTGCTTACGCGTTACTCACCCG
TTTGCCACTCGAATTGATAAGAAGCAAGCTTCTCATCTCTTCTCGTTCGACTGCA
(consensus 16S rRNA sequence for the Megasphaera strain deposited
under accession number NCIMB 43387)
SEQ ID NO: 7
TCGAACGGCTGGTTCCTTGCGGTTGCCTCACCGGCTTCGGGTGTGAATGACTTTCGTGGT
GTGACGGGCGGTGTGTACAAGGCCCGGGAACGTATTCACCGCAGTATGCTGACCTGCGA
TTACTAGCGATTCCTGCTTCATGCAGGCGAGTTGCAGCCTGCAATCCGAACTGGGACTCT
GTTTTTGGGGTTTGCTCCGGATCGCTCCTTCGCTTCCCTCTATTAAGAGCCATTGTAGTAC
GTGTGTAGCCCAAGCCATAAGGGGCATGATGACTTGACGTCATCCCCGCCTTCCTCCGC
ATTGTCTGCGGCAGTCTCTTCTGAGTCCCCACCCTTAGTGCTGGCAACAGAAGATAGGG
GTTGCGCTCGTTGCGGGACTTAACCCAACATCTCACGACACGAGCTGACGACAGCCGTG
CACCACCTGTTTTCTTGTCTTCCGAAGAAGAACCGGAAATCTCTTTCCGTAGCAATCAAT
GTCAAGGCTTGGTAAGGTTCTTCGCGTTGCGTCGAATTAAACCACATACTCCACCGCTTG
TGCGGGCCCCCGTCAATTCCTTTGAGTTTCAGCCTTGCGGCCGTACTCCCCAGGCGGGAT
ACTTATTGCGTTAACTCCGGCACAGAAGGAGTCGATACCTCCTACACCTAGTATCCATCG
TTTACGGCCAGGACTACCGGGGTATCTAATCCCGTTTGCTCCCCTGGCTTTCGCGCCTCA
GCGTCAGTTGTCGTCCAGAAAGCCGCTTTCGCCACTGGTGTTCCTCCTAATATCTACGCA
TTTCACCGCTACACTAGGAATTCCGCTTTCCTCTCCGACACTCGAGCTTCACAGTTTCGG
TCCCCTCACGGGGTTAAGCCCCGCACTTTTAAGACCGACTTGCGATGCCGCCTGCGCGCC
CTTTACGCCCAATAATTCCGGACAACGCTTGCCACCTACGTATTACCGCGGCTGCTGGCA
CGTAGTTAGCCGTGGCTTTCTCTTACGGTACCGTCAGGGATAACGGGTATTGACCGCTAT
CCTGTTCGTCCCATATAACAGAACTTTACAACCCGAAGGCCGTCATCGTTCACGCGGCGT
TGCTCCGTCAGACTTTCGTCCATTGCGGAAGATTCCCCACTGCTGCCTCCCGTAGGAGTC
TGGGCCGTGTCTCAGTCCCAATGTGGCCGTTCATCCTCTCAGACCGGCTACTGATCGTCG
CCTTGGTGGGCCGTTACCCCTCCAACTAGCTAATCAGACGCAAGCCCCTCCTCCAGCGA
AAGCCCGAAGGCCTCCCTTTCTTCATCCCGTCATGCGGCGGAAAGAACGTATTCGGTATT
AGCAGCCGTTTCCAGCTGTTGTCCCCATCTGAAGGGCAGGTTGCTTACGCGTTACTCACC
CGTTTGCCACTCGAATTGATAAGAAGCAAGCTTCTCATCTCTTCTCGTTCGACTTGCA

### REFERENCES

[1] Spor et al. (2011) Nat Rev Microbiol. 9(4):279-90.
[2] Eckburg et al. (2005) Science. 10; 308(5728):1635-8.
[3] Macpherson et al. (2001) Microbes Infect. 3(12):1021-35
[4] Macpherson et al. (2002) Cell Mol Life Sci. 59(12):2088-96.
[5] Mazmanian et al. (2005) Cell 15; 122(1):107-18.
[6] Frank et al. (2007) PNAS 104(34):13780-5.
[7] Scanlan et al. (2006) J Clin Microbiol. 44(11):3980-8.
[8] Kang et al. (2010) Inflamm Bowel Dis. 16(12):2034-42.
[9] Machiels et al. (2013) Gut. 63(8):1275-83.
[10] WO 2013/050792
[11] WO 03/046580
[12] WO 2013/008039
[13] WO 2014/167338
[14] Goldin and Gorbach (2008) Clin Infect Dis. 46 Suppl 2:S96-100.
[15] Azad et al. (2013) BMJ. 347:f6471.
[16] Holt, J G et al (1994) Bergey's Manual of Determinative Bacteriology (9th Ed) Lippincott Williams & Wilkins, p 11
[17] Ferreyro et al (2014) Cell Host and Microbe 16: 770-7
[18] Smits et al (2016) Nat Rev Dis Primers 2: Article number 16020
[19] McDonald et al (2018) Gastroenterolgy https://doi.org/10.1053/j.gastro.2018.07.014
[20] Padmanabhan et al. (2013) Standards in Genomic Sciences 8:525-538
[21] Masco et al. (2003) Systematic and Applied Microbiology, 26:557-563
[22] Srůtková et al. (2011) J. Microbiol. Methods, 87(1):10-6.
[23] Spencer et al (2003). Journal of Clinical Pathology, 56(3), 182-7
[24] Forbes (2002). Bailey & Scott's Diagnostic Microbiology 11th ed.
[25] Miyamoto-Shinohara et al. (2008) J. Gen. Appl. Microbiol., 54, 9-24.
[26] Cryopreservation and Freeze-Drying Protocols, ed. by Day and McLellan, Humana Press.
[27] Leslie et al. (1995) Appl. Environ. Microbiol. 61, 3592-3597.
[28] Mitropoulou et al. (2013) J Nutr Metab. (2013) 716861.
[29] Kailasapathy et al. (2002) Curr Issues Intest Microbiol. 3(2):39-48.
[30] Handbook of Pharmaceutical Excipients, 2nd Edition, (1994), Edited by A Wade and PJ Weller
[31] Remington's Pharmaceutical Sciences, Mack Publishing Co. (A. R. Gennaro edit. 1985)
[32] US 2016/0067188
[33] Handbook of Microbiological Media, Fourth Edition (2010) Ronald Atlas, CRC Press.
[34] Maintaining Cultures for Biotechnology and Industry (1996) Jennie C. Hunter-Cevera, Academic Press
[35] Strobel (2009) Methods Mol Biol. 581:247-61.
[36] Gennaro (2000) Remington: The Science and Practice of Pharmacy. 20th edition, ISBN: 0683306472.
[37] Molecular Biology Techniques: An Intensive Laboratory Course, (Ream et al., eds., 1998, Academic Press).
[38] Methods In Enzymology (S. Colowick and N. Kaplan, eds., Academic Press, Inc.)
[39] Handbook of Experimental Immunology, Vols. I-IV (D. M. Weir and C. C. Blackwell, eds, 1986, Blackwell Scientific Publications)
[40] Sambrook et al. (2001) Molecular Cloning: A Laboratory Manual, 3rd edition (Cold Spring Harbor Laboratory Press).
[41] Handbook of Surface and Colloidal Chemistry (Birdi, K. S. ed., CRC Press, 1997)
[42] Ausubel et al. (eds) (2002) Short protocols in molecular biology, 5th edition (Current Protocols).
[43] PCR (Introduction to Biotechniques Series), 2nd ed. (Newton & Graham eds., 1997, Springer Verlag)
[44] Current Protocols in Molecular Biology (F. M. Ausubel et al., eds., 1987) Supplement 30
[45] Smith & Waterman (1981) Adv. Appl. Math. 2: 482-489.
[46] Johnsen et al (2017) Journal of Chromatography A. 1503: 57-64
[47] van Roy and Berx (2008) Cell Mol Life Sci 65(23):3756-3788
[48] Feldman et al (2005) Adv Drug Deliv Rev 57(6):883-917

1.-23. (canceled)

24. A method of treating a Gram-positive bacterial infection in a subject in need thereof, comprising administering to the subject a composition comprising a bacterial strain that produces valeric acid, and wherein the bacterial strain is not of the species Megasphaera elsdenii.

25. The method of claim 24, wherein the production of valeric acid by the bacterial strain is determined using gas chromatography/mass spectrometry (GC/MS).

26. The method of claim 24, wherein the Gram-positive bacterial infection is in the gastrointestinal tract.

27. The method of claim 24, wherein the Gram-positive bacterial infection is a pathogenic Gram-positive bacterial infection.

28. The method of claim 24, wherein the composition reduces the viability of the Gram-positive bacteria, when administered to the subject.

29. The method of claim 24, wherein the composition inhibits the growth of the Gram-positive bacteria, when administered to the subject.

30. The method of claim 24, wherein the Gram-positive bacterial infection is caused by bacteria of the genus Clostridium, Staphylococcus, Enterococcus spp, Bacillus, Erysipelothrix, or Listeria.

31. The method of claim 24, wherein the Gram-positive bacterial infection is a Clostridium difficile infection.

32. The method of claim 31, wherein the composition treats one or more conditions associated with the Clostridium difficile infection selected from the group consisting of diarrhea, abdominal pain, pyrexia, hematochezia, dehydration, weight loss, toxic megacolon, gastrointestinal perforation, abdominal distension, colonic distension, nausea, pseudomembranous colitis, multiple organ dysfunction syndrome, and sepsis.

33. The method of claim 24, wherein the subject has been administered or is being administered one or more antibiotics.

34. The method of claim 33, wherein the one or more antibiotics comprises a broad-spectrum antibiotic or is selected from the group consisting of Vancomycin, Bactrium, Doxycyline, Ceftobiprole, Ceftaroline, Clindamycin, Dalbavancin, Daptomycin, Fusidic acid, Linezolid, Mupirocin, Oritavancin, Tedizolid, Telavancin, Tigecycline, Aminoglycosides, Carbapenems, Ceftazidime, Cefepime, Ceftobiprole, Ceftolozane/tazobactam, Fluoroquinolones, Piperacillin/tazobactam, Ticarcillin/clavulanic acid, Linezolid, Streptogramins, Tigecycline, and Daptomycin.

35. The method of claim 24, wherein the bacterial strain is of the genus Megasphaera.

36. The method of claim 35, wherein the composition comprises no more than de minimis or biologically irrelevant amounts of bacteria from another genus.

37. The method of claim 35, wherein the composition does not contain bacteria from any other genus.

38. The method of claim 35, wherein the bacterial strain is of the species Megasphaera cerevisiae, Megasphaera massiliensis, Megasphaera indica, Megasphaera paucivorans, Megasphaera sueciensis, Megasphaera micronuciformis, or Megasphaera hexanoica.

39. The method of claim 24, wherein the bacterial strain comprises a 16S rRNA gene sequence that has at least 95% sequence identity to the polynucleotide sequence of SEQ ID NO: 1, 2, 4, 5, 6, or 7.

40. The method of claim 24, wherein the bacterial strain is the strain deposited at NCIMB under accession number NCIMB 42787, NCIMB 43385, NCIMB 43386, NCIMB 43387, NCIMB 43388, or NCIMB 43389.

41. A method of treating a Gram-positive bacterial infection in a subject in need thereof, comprising administering to the subject a composition comprising a bacterial strain that produces valeric acid, wherein the bacterial strain is of the species Megasphaera massiliensis.

42. The method of claim 41, wherein the bacterial strain comprises a 16S rRNA gene sequence that has at least 95% sequence identity to the polynucleotide sequence of SEQ ID NO: 1, 2, 4, or 5.

43. The method of claim 41, wherein the bacterial strain is the strain deposited at NCIMB under accession number NCIMB 42787, NCIMB 43388, or NCIMB 43389.
